(19) United States
(12) Patent Application Publication
(10) Pub. No.: US 2005/0266469 A1
(43) Pub. Date: Dec. 1, 2005

(54) Alternatively spliced isoforms of checkpoint kinase 1 (CHK1)

(76) Inventors: Christopher K. Raymond, Seattle, WA (US); Philip W. Garrett-Engele, Seattle, WA (US); John C. Castle, Seattle, WA (US)

Correspondence Address: R. Douglas Bradley;Merck & Co., Inc., Patent Department RY60-30, P.O. Box 2000, Rahway, NJ, 07065-0907, US

(21) Appl. No.: 11/137,315

(22) Filed: May 25, 2005

Related U.S. Application Data
(60) Provisional application No. 60/574,380, filed on May 25, 2004.

(51) Int. Cl. C12Q 1/68; C07H 21/04; C12P 21/06; C12N 9/12

(52) U.S. Cl. 435/6; 435/69.1; 435/194; 435/320.1; 435/325; 536/23.2

(57) ABSTRACT

The present invention features nucleic acids and polypeptides encoding two novel splice variant isoforms of checkpoint kinase 1 (CHK1). The polynucleotide sequences of CHK1sv1 and CHK1sv2 are provided by SEQ ID NO 3 and SEQ ID NO 5, respectively. The amino acid sequences for CHK1sv1 and CHK1sv2 are provided by SEQ ID NO 4 and SEQ ID NO 6, respectively. The present invention also provides methods for using CHK1sv1 and CHK1sv2 polynucleotides and proteins to screen for compounds that bind to CHK1sv1 and CHK1sv2, respectively.

This application claims priority to U.S. Provisional Patent Application Ser. No. 60/574,380 filed on May 25, 2004, which is incorporated by reference herein in its entirety.

### BACKGROUND OF THE INVENTION

The references cited herein are not admitted to be prior art to the claimed invention.

The cell cycle checkpoint kinase 1 (CHK1) plays an integral role in the checkpoint and DNA damage response pathways. Checkpoint pathways are signaling cascades which control progression through the cell cycle and help ensure genomic integrity. Upon DNA damage, the cell cycle is arrested to facilitate DNA repair or induce apoptosis. CHK1 also regulates genes involved in DNA repair, cell cycle arrest, apoptosis, chromatin remodeling, and stress-induced transcription. Defective checkpoints contribute to genomic instability and evolution of tumors; the function of CHK1 has been implicated in cancer (reviewed in Zhou and Elledge, 2000, Nature 408:433-439; Bartek and Lukas, 2003, Cancer Cell 3:421-429).

The somatic cell cycle is divided into several distinct phases. Nuclear division and cytokinesis take place during M phase, or mitotic phase. During interphase, the period between each M phase, cell growth occurs. Interphase consists of 3 subphases. Following mitosis is the G₁ phase, a period of biosynthesis. DNA replication then occurs during the S phase. Following the S phase is the G₂ phase, a safety gap before commitment to mitosis (Alberts et al., 1994, Molecular Biology of the Cell, 3^{rd} edition, Garland Publishing, New York). CHK1 regulates the G₂ and S-phase cell cycle checkpoints (reviewed in Zhou and Bartek, 2004, Nat. Rev. Cancer, 4:216-225).

The human CHK1 gene is localized on chromosome 11 (Sanchez et al., 1997, Science 277:1497-1501). The CHK1 mRNA transcript (NM_{—}001274) consists of 13 exons, of which the latter 12 encode a 476 amino acid protein. Exon 1 of the CHK1 mRNA transcript (NM_{—}00274) does not encode any amino acids. The CHK1 protein is composed of a kinase domain (amino acids 16-265), a putative flexible linker region, SQ domains (amino acids 305-325 and 340-356), and a C-terminal domain of undefined function (Sanchez et al., 1997, Science 277:1497-1501). CHK1 homologs have been identified in other species, and the human CHK1 protein has 92% identity to the mouse CHK1 and 94% identity to the rat CHK1 (Sanchez et al., 1997, Science 277:1497-1501; Shann and Hsu, 2001, J. Biol. Chem. 276: 48863-48870). Northern blot analysis indicated ubiquitous expression in human tissues, with particular abundance in thymus, testis, small intestine, and colon. Indirect immunofluoresence showed that CHK1 is localized to the nucleus (Sanchez et al., 1997, Science 277:1497-1501).

Upon DNA damage, unidentified sensors initiate signaling to signal transducers. ATM and ATR, members of the phophatidylinositol 3-kinase family, are key signal transducers of the DNA damage response pathway. ATM and ATR phosphorylate and activate the effector kinases CHK1 and CHK2, two serine/threonine kinases that are structurally unrelated but functionally overlapping. These checkpoint kinases regulate numerous pathways, such as cell cycle delay, apoptosis, transcription, and DNA repair (reviewed in Zhou and Elledge, 2000, Nature 408:433-439; Bartek and Lukas, 2003, Cancer Cell 3:421-429; Zhou and Bartek, 2004, Nat. Rev. Cancer, 4:216-225).

The ATM pathway is activated by ionizing radiation (IR), double-strand breaks, and alkylating agents. The ATR pathway responds to ultraviolet (UV), replication inhibitors, such as hydroxyurea, as well as and alkylating agents. Activated ATM phosphorylates CHK2 at the T68 residue within the SQ domains. Activated CHK2 may inhibit CDC25A causing S-phase delay, induce G₁-S delay through p53-mediated mechanisms, induce apoptosis through p53-dependent or independent (PML or E2F1) pathways, or modulate DNA repair through BRCA1. Phosphorylation/activation of CHK1 is largely mediated by ATR at residues S317 and S345 within the SQ domains (reviewed in Bartek and Lukas, 2003, Cancer Cell 3:421-429; Zhou and Bartek, 2004, Nature Rev. Cancer 4:1-10). However, there is some cross-talk between the ATM/ATR pathways, as ATM can also phosphorylate CHK1 at S317 in response to IR (Gatei et al., 2003, J. Biol. Chem. 278:14806-14811). Optimal activation of CHK1 may also require additional proteins, such as the tumor suppressor BRCA1 and claspin (Yarden et al., 2002, Nat. Genet. 30:285-289; Kumagai and Dunphy, 2000, Mol Cell 6: 839-849).

CHK1 is expressed largely in the S and G₂ phases of proliferating cells (Lukas et al., 2001, Cancer Res. 61:4990-4993). RNA interference experiments demonstrated that CHK1 mediates both S and G₂ checkpoints in mammalians following DNA damage (Zhao et al., 2002, Proc. Natl. Acad. Sci. USA 99:14795-14800; Xiao et al., 2003, J. Biol. Chem., 278:21767-21773). CHK1 phosphorylates CDC25A at residue S123, which inactivates the protein and targets it for ubiquitin-mediated degradation. As a result, CDK2 and CDC2 are not activated by CDC25A phosphatase activity, and the cell cycle arrests in late G₁, S, and G₂ phases (Mailand et al., 2000, Science 288:1425-1429; Zhao et al., 2002, Proc. Natl. Acad. Sci. USA 99:14795-14800; reviewed in Iliakis et al., 2003, Oncogene, 22:5834-5847; Zhou and Bartek, 2004, Nature Rev. Cancer 4:1-10). CHK1 can also negatively regulate CDC25C by phosphorylation of S216. 14-3-3 protein then can bind to the phosphorylated CDC25C, sequestering the complex in the cytoplasm. Consequently, CDC25C cannot dephosphorylate CDC2 at the key T14 and S15 residues, which prevents entry to mitosis (Peng et al., 1997, Science 277:1501-1505; reviewed in Iliakis et al., 2003, Oncogene, 22:5834-5847; Zhou and Bartek, 2004, Nature Rev. Cancer 4:1-10).

Recent work has demonstrated CHK1 autoinhibition. A kinase inhibitory domain in the C-terminus was suggested by Chen et al. (2000, Cell 100:681-692), as full-length CHK1 was 20-fold less active towards substrates than the CHK1 kinase domain (amino acids 1-265). Within Xenopus, this autoinhibitory region (AIR) in the CHK1 C-terminus, which overlaps with the nuclear localization signal, has been suggested to bind with the kinase domain of CHK1 in an intramolecular fashion. Interaction between the AIR and kinase domain is influenced by phosphorylation of the SQ domains (Katsuragi and Sagata, 2004, Mol. Biol. Cell. 15:1680-1689). A human CHK1 C-terminus deletion mutant (amino acids 1-389) also displayed autophosphorylation, high kinase activity to a CDC25C substrate, and induced cell cycle delay (Ng et al., 2004, J. Biol. Chem. 279:8808-8819).

Splice variants of mammalian CHK1 have been identified. Shann and Hsu (2001, J. Biol. Chem. 276:48863-48870) have isolated and characterized a rat liver-specific CHK1 isoform (Cil), whose protein sequence starts within the flexible linker region at amino acid 283 compared to the reference rat CHK1 protein (NP_{—}536325). The deletion of the kinase domain in Cil results from the use of an alternative promoter within intron 5 and use of an alternative exon 5b within intron 5. The C-terminal portion of CHK1 may act as a negative regulator of kinase activity (Chen et al., 2000, Cell 100:681-692). A yeast two-hybrid assay demonstrated that Cil could interact with the kinase domain of CHK1. Furthermore, Cil may act as a dominant negative competitor of CHK1, as demonstrated by its inhibition of p53 transactivation (Shann and Hsu, 2001, J. Biol. Chem. 276:48863-48870). A truncated CHK1 isoform has also been identified in human small cell lung cancer cells (Haruki et al., 2000, Cancer Res. 60:4689-4692). This human CHK1 isoform lacks amino acids encoded by exon 8 (amino acids 240-271), which corresponds to the conserved subdomain XI within the catalytic domain of CHK1 kinase (Haruki et al., 2000, Cancer Res. 60:4689-4692). Chen et al. (2000, Cell 100:681-692) has also suggested that the E residue at position 248 and the R residue at position 253, which are absent in this splice variant, may be involved anchoring of the activation loop of CHK1. However the functional significance of this human CHK1 isoform has not been determined (Haruki et al., 2000, Cancer Res. 60:4689-4692).

Consistent with the function of CHK1 in regulating cell cycle progression and DNA damage response pathways, mutations of CHK1 have been associated with cancer. Heterozygous frameshift mutations in adenine tracts within the coding region of CHK1 have been found in colon cancer cells, endometrial cancer cells, and stomach tumors (Bertoni et al., 1999, Genes Chromosomes Cancer, 26:176-180; Vassileva et al., 2002, Cancer Res. 62:4095-4099; Menoyo et al., 2001, Cancer Res. 61:7727-7730). The functional significance for the heterozygous CHK1 mutations has not yet been determined for human cancers.

Studies of CHK1 knockout mice have indicated that this gene is essential for embryonic growth and development. CHK1 deficiency resulted in early embryonic lethality, occurring during the 1^{st} week of gestation (Takai et al., 2000, Genes Dev., 14:1439-1447; Liu et al, 2000, Genes Dev., 14:1448-1459). CHK1^{-/-} blastocysts displayed aberrant nuclei, degeneration of the inner cell mass, and cell death by apoptosis; CHK1^{-/-} embryonic stem cells and embryos lacked cell cycle checkpoints in response to DNA damage (Takai et al., 2000, Genes Dev., 14:1439-1447; Liu et al, 2000, Genes Dev., 14:1448-1459). CHK1 heterozygous embryonic development appeared normal (Takai et al., 2000, Genes Dev., 14:1439-1447; Liu et al, 2000, Genes Dev., 14:1448-1459). However, CHK1 heterozygosity enhanced mammary tumor development in WNT-1 transgenic mice (Liu et al., 2000, Genes Dev. 14:1448-1459). In contrast to embryonic cells, CHK1 deficient avian DT40 cells are viable, but they do exhibit checkpoint defects in response to DNA damage (Zachos et al., 2003, EMBO J. 22:713-723).

Several compounds and peptides have been identified that inhibit CHK1. UCN-01 (7-hydroxystaurosporine) protein kinase antagonist has been found to be a potent G₂ checkpoint abrogator in p53 deficient cells, and enhanced destruction of these cells by the DNA-damaging agent, cisplatin (Wang et al., 1996 J. Natl. Cancer Instit. 88:956-965). It has been demonstrated that UCN-01 abrogation of the G₂ checkpoint was mediated by inhibition of CHK1 (Graves et al., 2000, J. Biol. Chem. 275:5600-5605; Busby et al., 2000, Cancer Res. 60:2108-2112). Other indolocarbazole inhibitors, such as SB-218078 and Gö6976, also prevented G₂ arrest in a CHK1-specific manner (Jackson et al., 2000, Cancer Res. 60:566-572; Kohn et al., 2003, Cancer Res. 63:31-35). Peptides corresponding to amino acids 211-221 of human CDC25C, fused to a portion of HIV1-TAT to facilitate heterologous protein uptake across the cell membrane, have also demonstrated the ability to inactivate CHK1 and abrogate the G₂ checkpoint (Suganuma et al., 1999, Cancer Res. 59:5887-5891). Recently a Schizosaccharomyces pombe protein DIS2 has been identified as an inactivator of CHK1 and may be involved in a checkpoint release pathway (Den Elzen and O'Connell, 2004, EMBO J. 23:908-918).

Targeting checkpoint pathways underlies the chemosensitization strategy for eliminating tumor cells. Tumor cells share characteristics such as genetic instability and rapid proliferation. While anticancer agents such as topoisomerase inhibitors or alkylating agents induce DNA damage and are effective drugs, they are accompanied by high toxicity, small therapeutic index, and considerable development of resistance. The rationale behind chemosensitization is that tumor cells which are defective in some checkpoints, such as p53, could be targeted by selective inhibition of the remaining checkpoints, thus enhancing the effects of DNA damaging drugs and driving tumor cells to apoptosis (reviewed in Zhou et al., 2004, Nat. Rev. Cancer 4:1-10; Bartek and Lukas, 2003, Cancer Cell 3:421-425). Consistent with this strategy, CDC25C derived peptide inhibitors of CHK1 have been found to sensitize p53-deficient cancer cell lines to bleomycin treatment, but not affect cytotoxicity to normal cells (Suganuma et al., 1999, Cancer Res., 59:5887-5891). Inhibition of CHK1 function in p53-deficient tumor cells by RNAi or a dominant negative form of CHK1 also enhanced the cell-killing effects of doxorubicin or ionizing radiation, respectively (Chen et al., 2003, Mol. Cancer Ther. 2:543-548; Koniaras et al., 2001, Oncogene 20:7453-7463).

Because of the multiple therapeutic values of drugs targeting the CHK1 pathway, and the essential DNA damage responses regulated by CHK1, there is a need in the art to identify new isoform variants of CHK1 and for compounds that selectively bind to isoforms of CHK1. The present invention is directed towards two novel CHK1 isoforms (CHK1sv1 and CHK1sv2) and uses thereof.

### BRIEF DESCRIPTION OF THE FIGURES

FIG. 1A illustrates the exon structure of CHK1 mRNA corresponding to the known long reference form of CHK1 mRNA (labeled NM_{—}001274) and the exon structure corresponding to the inventive short form splice variants (labeled CHK1sv1 and CHK1sv2). FIG. 1B depicts the nucleotide sequences of the exon junctions resulting from the splicing of exon 9 to exon 11 (SEQ ID NO 1) in the case of CHK1sv1 mRNA and the splicing of exon 2 to exon 4 (SEQ ID NO 2) in the case of CHK1sv2 mRNA. In FIG. 1B, in the case of CHK1sv1, the nucleotides shown in italics represent the 20 nucleotides at the 3′ end of exon 9 and the nucleotides shown in underline represent the 20 nucleotides at the 5′ end of exon 11 and in the case of CHK1sv2, the nucleotides shown in italics represent the 20 nucleotides at the 3′ end of exon 2 and the nucleotides shown in underline represent the 20 nucleotides at the 5′ end of exon 4.

### SUMMARY OF THE INVENTION

Real-time PCR experiments and RT-PCR have been used to identify and confirm the presence of novel splice variants of human CHK1 mRNA. More specifically, the present invention features polynucleotides encoding different protein isoforms of CHK1. A polynucleotide sequence encoding CHK1sv1 is provided by SEQ ID NO 3. An amino acid sequence for CHK1sv1 is provided by SEQ ID NO 4. A polynucleotide sequence encoding CHK1sv2 is provided by SEQ ID NO 5. An amino acid sequence for CHK1sv2 is provided by SEQ ID NO 6.

Thus, a first aspect of the present invention describes a purified CHK1sv1 encoding nucleic acid and a purified CHK1sv2 encoding nucleic acid. The CHK1sv1 encoding nucleic acid comprises SEQ ID NO 3 or the complement thereof. The CHK1sv2 encoding nucleic acid comprises SEQ ID NO 5 or the complement thereof. Reference to the presence of one region does not indicate that another region is not present. For example, in different embodiments the inventive nucleic acid can comprise, consist, or consist essentially of an encoding nucleic acid sequence of SEQ ID NO 3 or alternatively can comprise, consist, or consist essentially of the nucleic acid sequence of SEQ ID NO 5.

Another aspect of the present invention describes a purified CHK1sv1 polypeptide that can comprise, consist or consist essentially of the amino acid sequence of SEQ ID NO 4. An additional aspect describes a purified CHK1sv2 polypeptide that can comprise, consist, or consist essentially of the amino acid sequence of SEQ ID NO 6.

Another aspect of the present invention describes expression vectors. In one embodiment of the invention, the inventive expression vector comprises a nucleotide sequence encoding a polypeptide comprising, consisting, or consisting essentially of SEQ ID NO 4, wherein the nucleotide sequence is transcriptionally coupled to an exogenous promoter. In another embodiment, the inventive expression vector comprises a nucleotide sequence encoding a polypeptide comprising, consisting, or consisting essentially of SEQ ID NO 6, wherein the nucleotide sequence is transcriptionally coupled to an exogenous promoter.

Alternatively, the nucleotide sequence comprises, consists, or consists essentially of SEQ ID NO 3, and is transcriptionally coupled to an exogenous promoter. In another embodiment, the nucleotide sequence comprises, consists, or consists essentially of SEQ ID NO 5, and is transcriptionally coupled to an exogenous promoter.

Another aspect of the present invention describes recombinant cells comprising expression vectors comprising, consisting, or consisting essentially of the above-described sequences and the promoter is recognized by an RNA polymerase present in the cell. Another aspect of the present invention describes a recombinant cell made by a process comprising the step of introducing into the cell an expression vector comprising a nucleotide sequence comprising, consisting, or consisting essentially of SEQ ID NO 3 or SEQ ID NO 5, or a nucleotide sequence encoding a polypeptide comprising, consisting, or consisting essentially of an amino acid sequence of SEQ ID NO 4 or SEQ ID NO 6, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing CHK1sv1 or CHK1sv2 polypeptide comprising SEQ ID NO 4 or SEQ ID NO 6, respectively. The method involves the step of growing a recombinant cell containing an inventive expression vector under conditions wherein the polypeptide is expressed from the expression vector.

Another aspect of the present invention features a purified antibody preparation comprising an antibody that binds selectively to CHK1sv1 as compared to one or more CHK1 isoform polypeptides that are not CHK1sv1. In another embodiment, a purified antibody preparation is provided comprising antibody that binds selectively to CHK1sv2 as compared to one or more CHK1 isoform polypeptides that are not CHK1sv2.

Another aspect of the present invention provides a method of screening for a compound that binds to CHK1sv1, CHK1sv2 or fragments thereof. In one embodiment, the method comprises the steps of: (a) expressing a polypeptide comprising the amino acid sequence of SEQ ID NO 4 or a fragment thereof from recombinant nucleic acid; (b) providing to said polypeptide a labeled CHK1 ligand that binds to said polypeptide and a test preparation comprising one or more test compounds; (c) and measuring the effect of said test preparation on binding of said test preparation to said polypeptide comprising SEQ ID NO 4. Alternatively, this method could be performed using SEQ ID NO 6 instead of SEQ ID NO 4.

In another embodiment of the method, a compound is identified that binds selectively to CHK1sv1 polypeptide as compared to one or more CHK1 isoform polypeptides that are not CHK1sv1. This method comprises the steps of: providing a CHK1sv1 polypeptide comprising SEQ ID NO 4; providing a CHK1 isoform polypeptide that is not CHK1sv1; contacting said CHK1sv1 polypeptide and said CHK1 isoform polypeptide that is not CHK1sv1 with a test preparation comprising one or more test compounds; and determining the binding of said test preparation to said CHK1sv1 polypeptide and to CHK1 isoform polypeptide that is not CHK1sv1, wherein a test preparation that binds to said CHK1sv1 polypeptide but does not bind to said CHK1 isoform polypeptide that is not CHK1sv1 contains a compound that selectively binds said CHK1sv1 polypeptide. Alternatively, the same method can be performed using CHK1sv2 polypeptide comprising, consisting, or consisting essentially of SEQ ID NO 6.

In another embodiment of the invention, a method is provided for screening for a compound able to bind to or interact with a CHK1sv1 protein or a fragment thereof comprising the steps of: expressing a CHK1sv1 polypeptide comprising SEQ ID NO 4 or a fragment thereof from a recombinant nucleic acid; providing to said polypeptide a labeled CHK1 ligand that binds to said polypeptide and a test preparation comprising one or more compounds; and measuring the effect of said test preparation on binding of said labeled CHK1 ligand to said polypeptide, wherein a test preparation that alters the binding of said labeled CHK1 ligand to said polypeptide contains a compound that binds to or interacts with said polypeptide. In an alternative embodiment, the method is performed using CHK1sv2 polypeptide comprising, consisting, or consisting essentially of SEQ ID NO 6 or a fragment thereof.

Another aspect of the present invention provides a method of screening for a compound that binds to one or more CHK1 isoform polypeptides that are not CHK1sv1. This method comprises the steps of: providing a CHK1sv1 polypeptide comprising SEQ ID NO 4; providing a CHK1 isoform polypeptide that is not CHK1sv1; contacting said CHK1sv1 polypeptide and CHK1 isoform polypeptide that is not CHK1sv1 with a test preparation comprising one or more test compounds; and determining the binding of said test preparation to said CHK1sv1 polypeptide and to said CHK1 isoform polypeptide that is not CHK1sv1, wherein a test preparation that binds to said CHK1 isoform polypeptide that is not CHK1sv1 but not to said CHK1sv1 polypeptide contains a compound that selectively binds said CHK1 isoform polypeptide. Alternatively, the same method can be performed using CHK1sv2 polypeptide comprising, consisting, or consisting essentially of SEQ ID NO 6.

Other features and advantages of the present invention are apparent from the additional descriptions provided herein, including the different examples. The provided examples illustrate different components and methodology useful in practicing the present invention. The examples do not limit the claimed invention. Based on the present disclosure the skilled artisan can identify and employ other components and methodology useful for practicing the present invention.

Definitions

Unless defined otherwise, all technical and scientific terms used herein have the meaning commonly understood by one of ordinary skill in the art to which this invention belongs.

As used herein, “CHK1” refers to human checkpoint kinase 1 protein (CHK1), also known as CHEK1, (NP_{—}001265). In contrast, reference to a CHK1 isoform, includes NP_{—}001265 and other polypeptide isoform variants of CHK1.

As used herein, “CHK1sv1” and “CHK1sv2” refer to splice variant isoforms of human CHK1 protein, wherein the splice variants have the amino acid sequence set forth in SEQ ID NO 4 (for CHK1sv1) and SEQ ID NO 6 (for CHK1sv2).

As used herein, “CHK1” refers to polynucleotides encoding CHK1.

As used herein, “CHK1sv1” refers to polynucleotides encoding CHK1sv1 having an amino acid sequence set forth in SEQ ID NO 4. As used herein, “CHK1sv2” refers to polynucleotides encoding CHK1sv2 having an amino acid sequence set forth in SEQ ID NO 6.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect to the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv(scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

The present invention relates to the nucleic acid sequences encoding human CHK1sv1 and CHK1sv2 that are alternatively spliced isoforms of CHK1, and to the amino acid sequences encoding these proteins. SEQ ID NO 3 and SEQ ID NO 5 are polynucleotide sequences representing exemplary open reading frames that encode the CHK1sv1 and CHK1sv2 proteins, respectively. SEQ ID NO 4 shows the polypeptide sequence of CHK1sv1. SEQ ID NO 6 shows the polypeptide sequence of CHK1sv2.

CHK1sv1 and CHK1sv2 polynucleotide sequences encoding CHK1sv1 and CHK1sv2 proteins, as exemplified and enabled herein include a number of specific, substantial and credible utilities. For example, CHK1sv1 and CHK1sv2 encoding nucleic acids were identified in a RNA sample obtained from a human source (see Examples 1 and 2). Such nucleic acids can be used as hybridization probes to distinguish between cells that produce CHK1sv1 and CHK1sv2 transcripts from human or non-human cells (including bacteria) that do not produce such transcripts. Similarly, antibodies specific for CHK1sv1 or CHK1sv2 can be used to distinguish between cells that express CHK1sv1 or CHK1sv2 from human or non-human cells (including bacteria) that do not express CHK1sv1 or CHK1sv2.

CHK1 is an important drug target for the management of cancer (reviewed in Zhou and Bartek, 2004, Nat. Rev. Cancer 4:1-10; Bartek and Lukas, 2003, Cancer Cell 3:421-429). Given the potential importance of CHK1 activity to the therapeutic management of cancer, it is of value to identify CHK1 isoforms and identify CHK1-ligand compounds that are isoform specific, as well as compounds that are effective ligands for two or more different CHK1 isoforms. In particular, it may be important to identify compounds that are effective inhibitors of a specific CHK1 isoform activity, yet do not bind to or interact with a plurality of different CHK1 isoforms. Compounds that bind to or interact with multiple CHK1 isoforms may require higher drug doses to saturate multiple CHK1-isoform binding sites and thereby result in a greater likelihood of secondary non-therapeutic side effects. Furthermore, biological effects could also be caused by the interaction of a drug with the CHK1sv1 or CHK1sv2 isoforms specifically. For the foregoing reasons, CHK1sv1 and CHK1sv2 proteins represent useful compound binding targets and have utility in the identification of new CHK1-ligands exhibiting a preferred specificity profile and having greater efficacy for their intended use.

In some embodiments, CHK1sv1 and CHK1sv2 activity is modulated by a ligand compound to achieve one or more of the following: prevent or reduce the risk of occurrence of cancer. Compounds that treat cancers are particularly important because of the cause-and-effect relationship between cancers and mortality (National Cancer Institute's Cancer Mortality Rates Registry, http:H1www3.cancer.gov/atlasplus/charts.html, last visited Sep. 24, 2003).

Compounds modulating CHK1sv1 or CHK1sv2 include agonists, antagonists, and allosteric modulators. Inhibitors of CHK1 achieve clinical efficacy by a number of known and unknown mechanisms. While not wishing to be limited to any particular theory of therapeutic efficacy, generally, but not always, CHK1sv1 or CHK1sv2 compounds will be used to modulate kinase activity. UCN-01 and indolocarbazole inhibitors, such as SB-218078 and Gö6976, may act as inhibitors of G₂ arrest by blocking CHK1 activity (Busby et al., 2000, Cancer Res. 60:2108-2112; Jackson et al., 2000, Cancer Res. 60:566-572; Kohn et al., 2003, Cancer Res. 63:31-35). Peptides with sequences corresponding to amino acids 211-221 of human CDC25C have been used as CHK1 kinase inhibitors (Suganuma et al., 1999, Cancer Res. 59:5887-5891). Therefore, agents that modulate CHK1 activity may be used to achieve a therapeutic benefit for any disease or condition due to, or exacerbated by, CHK1 activity.

CHK1sv1 or CHK1sv2 activity can also be affected by modulating the cellular abundance of transcripts encoding CHK1sv1 or CHK1sv2, respectively. Compounds modulating the abundance of transcripts encoding CHK1sv1 or CHK1sv2 include a cloned polynucleotide encoding CHK1sv1 or CHK1sv2, respectively, that can express CHK1sv1 or CHK1sv2 in vivo, antisense nucleic acids targeted to CHK1sv1 or CHK1sv2 transcripts, enzymatic nucleic acids, such as ribozymes, and RNAi nucleic acids, such as shRNAs or siRNAs, targeted to CHK1sv1 or CHK1sv2 transcripts.

In some embodiments, CHK1sv1 or CHK1sv2 activity is modulated to achieve a therapeutic effect upon diseases in which regulation of CHK1 is desirable. For example, cancer may be treated by modulating CHK1sv1 or CHK1sv2 activities to inhibit activation of genes involved in DNA damage response pathways, in conjunction with other anticancer, DNA-damaging agents.

CHK1sv1 and CHK1sv2 Nucleic Acids

CHK1sv1 nucleic acids contain regions that encode for polypeptides comprising, consisting, or consisting essentially of SEQ ID NO 4. CHK1sv2 nucleic acids contain regions that encode for polypeptides comprising, consisting, or consisting essentially of SEQ ID NO 6. The CHK1sv1 and CHK1sv2 nucleic acids have a variety of uses, such as use as a hybridization probe or PCR primer to identify the presence of CHK1sv1 or CHK1sv2 nucleic acids, respectively; use as a hybridization probe or PCR primer to identify nucleic acids encoding for proteins related to CHK1sv1 or CHK1sv2, respectively; and/or use for recombinant expression of CHK1sv1 or CHK1sv2 polypeptides, respectively. In particular, CHK1sv1 polynucleotides do not have the polynucleotide region that consists of exon 10 of the CHK1 gene. CHK1sv2 polynucleotides do not have the polynucleotide region that consists of exon 3 of the CHK1 gene.

Regions in CHK1sv1 or CHK1sv2 nucleic acid that do not encode for CHK1sv1 or CHK1sv2, or are not found in SEQ ID NO 3 or SEQ ID NO 5, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding CHK1sv1 or CHK1sv2 related proteins from different sources. Obtaining nucleic acids encoding CHK1sv1 or CHK1sv2 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

CHK1sv1 or CHK1sv2 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:
A=Ala=Alanine: codons GCA, GCC, GCG, GCU
C=Cys=Cysteine: codons UGC, UGU
D=Asp=Aspartic acid: codons GAC, GAU
E=Glu=Glutamic acid: codons GAA, GAG
F=Phe=Phenylalanine: codons WUC, UUU
G=Gly=Glycine: codons GGA, GGC, GGG, GGU
H=His=Histidine: codons CAC, CAU
I=Ile=Isoleucine: codons AUA, AUC, AUU
K=Lys=Lysine: codons AAA, AAG
L=Leu=Leucine: codons UUA, UUG, CUA, CUC, CUG, CUU
M=Met=Methionine: codon AUG
N=Asn=Asparagine: codons AAC, AAU
P=Pro=Proline: codons CCA, CCC, CCG, CCU
Q=Gln=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr=Threonine: codons ACA, ACC, ACG, ACU
V=Val=Valine: codons GUA, GUC, GUG, GUU
W=Trp=Tryptophan: codon UGG
Y=Tyr=Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

CHK1sv1 and CHK1sv2 Probes

Probes for CHK1sv1 or CHK1sv2 contain a region that can specifically hybridize to CHK1sv1 or CHK1sv2 target nucleic acids, respectively, under appropriate hybridization conditions and can distinguish CHK1sv1 or CHK1sv2 nucleic acids from each other and from non-target nucleic acids, in particular CHK1 polynucleotides containing exon 10 and exon 3. Probes for CHK1sv1 or CHK1sv2 can also contain nucleic acid regions that are not complementary to CHK1sv1 or CHK1sv2 nucleic acids.

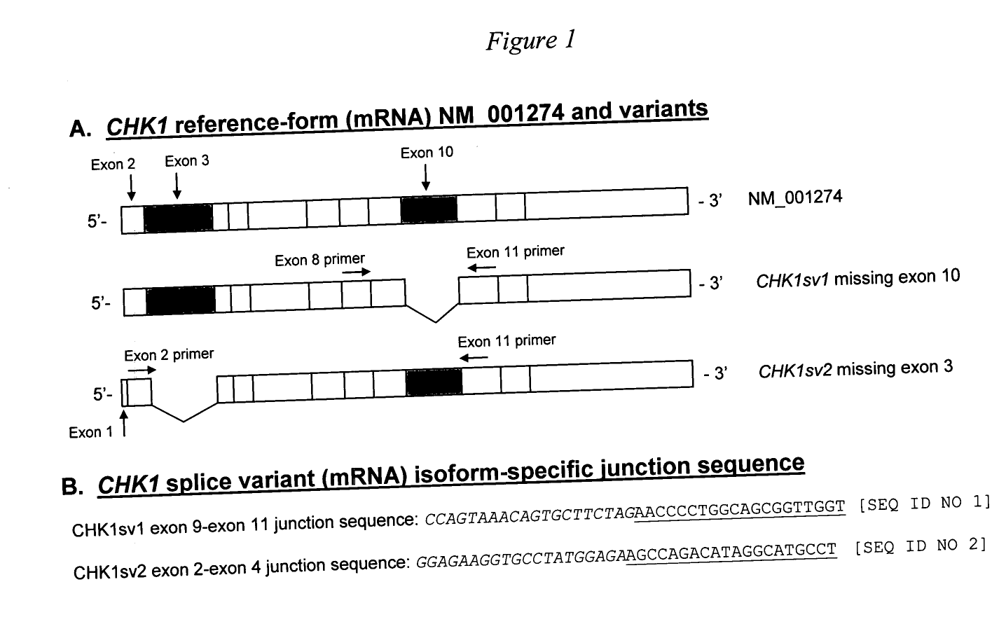

In embodiments where, for example, CHK1sv1 or CHK1sv2 polynucleotide probes are used in hybridization assays to specifically detect the presence of CHK1sv1 or CHK1sv2 polynucleotides in samples, the CHK1sv1 or CHK1sv2 polynucleotides comprise at least 20 nucleotides of the CHK1sv1 or CHK1sv2 sequence that correspond to the respective novel exon junction polynucleotide regions. In particular, for detection of CHK1sv1, the probe comprises at least 20 nucleotides of the CHK1sv1 sequence that corresponds to an exon junction polynucleotide created by the alternative splicing of exon 9 to exon 11 of the primary transcript of the CHK1 gene (see FIGS. 1A and 1B). For example, the polynucleotide sequence: 5′ GTGCTTCTAGAACCCCTGGC 3′ (SEQ ID NO 7) represents one embodiment of such an inventive CHK1sv1 polynucleotide wherein a first 10 nucleotide region is complementary and hybridizable to the 3′ end of exon 9 of the CHK1 gene and a second 10 nucleotide region is complementary and hybridizable to the 5′ end of exon 11 of the CHK1 gene (see FIG. 1B).

In another embodiment, for detection of CHK1sv2, the probe comprises at least 20 nucleotides of the CHK1sv2 sequence that corresponds to an exon junction polynucleotide created by the alternative splicing of exon 2 to exon 4 of the primary transcript of the CHK1 gene (see FIG. 1B). For example, the polynucleotide sequence: 5′CCTATGGAGAAGCCAGACAT 3′ (SEQ ID NO 8) represents one embodiment of such an inventive CHK1sv2 polynucleotide wherein a first 10 nucleotides region is complementary and hybridizable to the 3′ end of exon 2 of the CHK1 gene and a second 10 nucleotide region is complementary and hybridizable to the 5′ end of exon 4 of the CHK1 gene (see FIG. 1B).

In some embodiments, the first 20 nucleotides of a CHK1sv1 probe comprise a first continuous region of 5 to 15 nucleotides that is complementary and hybridizable to the 3′ end of exon 9 and a second continuous region of 5 to 15 nucleotides that is complementary and hybridizable to the 5′ end of exon 11. In some embodiments, the first 20 nucleotides of a CHK1sv2 probe comprise a first continuous region of 5 to 15 nucleotides that is complementary and hybridizable to the 3′ end of exon 2 and a second continuous region of 5 to 15 nucleotides that is complementary and hybridizable to the 5′ end of exon 4.

In other embodiments, the CHK1sv1 or CHK1sv2 polynucleotide comprises at least 40, 60, 80 or 100 nucleotides of the CHK1sv1 or CHK1sv2 sequence, respectively, that correspond to a junction polynucleotide region created by the alternative splicing of exon 9 to exon 11 in the case of CHK1sv1 or in the case of CHK1sv2, by the alternative splicing of exon 2 to exon 4 of the primary transcript of the CHK1 gene. In embodiments involving CHK1sv1, the CHK1sv1 polynucleotide is selected to comprise a first continuous region of at least 5 to 15 nucleotides that is complementary and hybridizable to the 3′ end of exon 9 and a second continuous region of at least 5 to 15 nucleotides that is complementary and hybridizable to the 5′ end of exon 11. Similarly, in embodiments involving CHK1sv2, the CHK1sv2 polynucleotide is selected to comprise a first continuous region of at least 5 to 15 nucleotides that is complementary and hybridizable to the 3′ end of exon 2 and a second continuous region of at least 5 to 15 nucleotides that is complementary and hybridizable to the 5′ end of exon 4. As will be apparent to a person of skill in the art, a large number of different polynucleotide sequences from the region of the exon 9 to exon 11 splice junction and the exon 2 to exon 4 splice junction may be selected which will, under appropriate hybridization conditions, have the capacity to detectably hybridize to CHK1sv1 or CHK1sv2 polynucleotides, respectively, and yet will hybridize to a much less extent or not at all to CHK1 isoform polynucleotides wherein exon 9 is not spliced to exon 11 or wherein exon 2 is not spliced to exon 4, respectively.

Preferably, non-complementary nucleic acid that is present has a particular purpose such as being a reporter sequence or being a capture sequence. However, additional nucleic acid need not have a particular purpose as long as the additional nucleic acid does not prevent the CHK1sv1 or CHK1sv2 nucleic acid from distinguishing between target polynucleotides, e.g., CHK1sv1 or CHK1sv2 polynucleotides, and non-target polynucleotides, including, but not limited to CHK1 polynucleotides not comprising the exon 9 to exon 11 splice junction or the exon 2 to exon 4 splice junctions found in CHK1sv1 or CHK1sv2, respectively.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Recombinant Expression

CHK1sv1 or CHK1sv2 polynucleotides, such as those comprising SEQ ID NO 3 or SEQ ID NO 5, respectively, can be used to make CHK1sv1 or CHK1sv2 polypeptides, respectively. In particular, CHK1sv1 or CHK1sv2 polypeptides can be expressed from recombinant nucleic acids in a suitable host or in vitro using a translation system. Recombinantly expressed CHK1sv1 or CHK1sv2 polypeptides can be used, for example, in assays to screen for compounds that bind CHK1sv1 or CHK1sv2, respectively. Alternatively, CHK1sv1 or CHK1sv2 polypeptides can also be used to screen for compounds that bind to one or more CHK1 isoforms, but do not bind to CHK1sv1 or CHK1sv2, respectively.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXT1 (Stratagene), pSG5 (Stratagene), pCMVLacl (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460). Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pRS416 (ATCC 87521), pPICZ (Invitrogen), pYES2 (Invitrogen), and Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) MRC-5 (ATCC CCL 171), and HEK 293 cells (ATCC CRL-1573).

Recombinant DNA molecules that feature precise fusions of polynucleotide sequences can also be assembled using standard recombinational subcloning techniques. Recombination-mediated, PCR-directed, or PCR-independent plasmid construction in yeast is well known in the art (see Hua et al., 1997, Plasmid 38:91-96; Hudson et al., 1997, Genome Res. 7:1169-1173; Oldenburg et al., 1997, Nucleic Acids Res. 25:451-452; Raymond et al., 1999, BioTechniques 26:134-8, 140-1). Overlapping sequences between the donor DNA fragments and the acceptor plasmid permit recombination in yeast. An example of recombination-mediated plasmid construction in Saccharomyces cerevisiae is described in Oldenburg et al. (1997, Nucleic Acids Res. 25:451-452): a DNA segment of interest was amplified by PCR so that the PCR product had 20-40 bp of homology at each end to the region of the plasmid at which recombination was to occur. The PCR product and linearized plasmid were co-transformed into yeast, and recombination resulted in replacement of the region between the homologous sequences on the plasmid with the region carried by the PCR fragment. The recombinational method of plasmid construction bypasses the need for extensive modification and ligation steps and does not rely on available restriction sites. These cloning vectors can then be utilized for protein expression in multiple systems.

In another embodiment of the invention, nucleic acid sequences encoding CHK1sv1 or CHK1sv2 may be ligated to a heterologous sequence resulting in translation of a fusion protein in any of the aforementioned host systems. For example, a chimeric CHK1sv1 or CHK1sv2 protein containing a heterologous moiety that can be recognized by a commercially available antibody may facilitate the screening of peptide libraries for inhibitors of CHK1sv1 or CHK1sv2 activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the CHK1sv1 or CHK1sv2 encoding sequence and the heterologous protein sequence, so that CHK1sv1 or CHK1sv2, respectively, may be cleaved away from the heterologous moiety following purification. Methods for fusion protein expression and purification are discussed in Ausubel (Current Protocols in Molecular Biology, John Wiley, 1987-1998). A variety of commercially available kits may also be used to facilitate expression and purification of fusion proteins.

To enhance expression in a particular host it may be useful to modify the sequence provided in SEQ ID NO 3 or SEQ ID NO 5 to take into account codon usage of the host. Codon usages of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

CHK1sv1 and CHK1sv2 Polypeptides

CHK1sv1 polypeptides contain an amino acid sequence comprising, consisting or consisting essentially of SEQ ID NO 4. CHK1sv2 polypeptides contain an amino acid sequence comprising, consisting or consisting essentially of SEQ ID NO 6. CHK1sv1 or CHK1sv2 polypeptides have a variety of uses, such as providing a marker for the presence of CHK1sv1 or CHK1sv2, respectively; use as an immunogen to produce antibodies binding to CHK1sv1 or CHK1sv2, respectively; use as a target to identify compounds binding selectively to CHK1sv1 or CHK1sv2, respectively; or use in an assay to identify compounds that bind to one or more isoforms of CHK1 but do not bind to or interact with CHK1sv1 or CHK1sv2, respectively.

In chimeric polypeptides containing one or more regions from CHK1sv1 or CHK1sv2 and one or more regions not from CHK1sv1 or CHK1sv2, respectively, the region(s) not from CHK1sv1 or CHK1sv2, respectively, can be used, for example, to achieve a particular purpose or to produce a polypeptide that can substitute for CHK1sv1 or CHK1sv2, or fragments thereof. Particular purposes that can be achieved using chimeric CHK1sv1 or CHK1sv2 polypeptides include providing a marker for CHK1sv1 or CHK1sv2 activity, respectively, enhancing an immune response, and altering the activity and regulation of CHK1.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional CHK1sv1 and CHK1sv2

Functional CHK1sv1 and CHK1sv2 are different protein isoforms of CHK1. The identification of the amino acid and nucleic acid sequences of CHK1sv1 or CHK1sv2 provide tools for obtaining functional proteins related to CHK1sv1 or CHK1sv2, respectively, from other sources, for producing CHK1sv1 or CHK1sv2 chimeric proteins, and for producing functional derivatives of SEQ ID NO 4 or SEQ ID NO 6.

CHK1sv1 or CHK1sv2 polypeptides can be readily identified and obtained based on their sequence similarity to CHK1sv1 (SEQ ID NO 4) or CHK1sv2 (SEQ ID NO 6), respectively. In particular, the CHK1sv1 polypeptide lacks a 178 base pair region corresponding to exon 10 of the CHK1 gene. The absence of exon 10 and the splicing of exon 9 to exon 11 of the CHK1 hnRNA transcript results in a shift of the protein reading frame at the exon 9 to exon 11 splice junction, thereby creating a carboxy terminal peptide region that is unique to the CHK1sv1 polypeptide as compared to the CHK1 reference sequence (NP_{—}001265). The frameshift creates a premature termination codon 29 nucleotides downstream of the exon 9/exon 11 splice junction. Therefore, CHK1sv1 polypeptide lacks an internal 59 amino acid region corresponding to the amino acid region encoded by exon 10 and is also lacking the amino acids encoded by the nucleotides downstream of the premature stop codon as compared to the CHK1 reference sequence (NP_{—}001265). CHK1sv2 lacks a 224 base pair region corresponding to exon 3 of the CHK1 gene. The CHK1sv2 polypeptide also initiates at an upstream AUG located at the end of exon 1, resulting in a shift of the protein reading frame as compared to the CHK1 reference transcript (NM_{—}001274). Initiation at an upstream AUG of a bicistronic RNA is a fairly common event and can be associated with disease (Meijer and Thomas, 2002 Biochem. J., 367:1-11; Kozak, 2002, Mamm. Genome 13:401-410). The use of the upstream AUG site, absence of exon 3, and resulting frameshift create an amino terminal peptide region that is unique to CHK1sv2 polypeptide as compared to the CHK1 reference sequence (NP_{—}001265).

Both the amino acid and nucleic acid sequences of CHK1sv1 or CHK1sv2 can be used to help identify and obtain CHK1sv1 or CHK1sv2 polypeptides, respectively. For example, SEQ ID NO 3 can be used to produce degenerative nucleic acid probes or primers for identifying and cloning nucleic acid polynucleotides encoding for a CHK1sv1 polypeptide. In addition, polynucleotides comprising, consisting, or consisting essentially of SEQ ID NO 3 or fragments thereof, can be used under conditions of moderate stringency to identify and clone nucleic acids encoding CHK1sv1 polypeptides from a variety of different organisms. The same methods can also be performed with polynucleotides comprising, consisting, or consisting essentially of SEQ ID NO 5, or fragments thereof, to identify and clone nucleic acids encoding CHK1sv2.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with CHK1sv1 or CHK1sv2 obtained from a particular source, derivatives can be produced. Such derivatives include polypeptides with amino acid substitutions, additions and deletions. Changes to CHK1sv1 or CHK1sv2 to produce a derivative having essentially the same properties should be made in a manner not altering the tertiary structure of CHK1sv1 or CHK1sv2, respectively.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

CHK1sv1 and CHK1sv2 Antibodies

Antibodies recognizing CHK1sv1 or CHK1sv2 can be produced using a polypeptide containing SEQ ID NO 4 in the case of CHK1sv1 or SEQ ID NO 6 in the case of CHK1sv2, respectively, or a fragment thereof, as an immunogen. Preferably, a CHK1sv1 polypeptide used as an immunogen consists of a polypeptide of SEQ ID NO 4 or a SEQ ID NO 4 fragment having at least 10 contiguous amino acids in length corresponding to the polynucleotide region representing the junction resulting from the splicing of exon 9 to exon 11 of the CHK1 gene. Preferably, a CHK1sv2 polypeptide used as an immunogen consists of a polypeptide derived from SEQ ID NO 6 or a SEQ ID NO 6 fragment, having at least 10 contiguous amino acids in length corresponding to a polynucleotide region representing the junction resulting from the splicing of exon 2 to exon 4 of the CHK1 gene.

In some embodiments where, for example, CHK1sv1 polypeptides are used to develop antibodies that bind specifically to CHK1sv1 and not to other isoforms of CHK1, the CHK1sv1 polypeptides comprise at least 10 amino acids of the CHK1sv1 polypeptide sequence corresponding to a junction polynucleotide region created by the alternative splicing of exon 9 to exon 11 of the primary transcript of the CHK1 gene (see FIG. 1). For example, the amino acid sequence: amino terminus-VNSASRTPGS-carboxy terminus (SEQ ID NO 9), represents one embodiment of such an inventive CHK1sv1 polypeptide wherein a first 5 amino acid region is encoded by a nucleotide sequence at the 3′ end of exon 9 of the CHK1 gene, the 6^{th} amino acid is encoded by nucleotide sequence at the exon 9/exon 11 splice junction, and a carboxy terminal 4 amino acid region is encoded by a nucleotide sequence at the 5′ end of exon 11 of the CHK1 gene. Preferably, at least 10 amino acids of the CHK1sv1 polypeptide comprises a first continuous region of 2 to 8 amino acids that is encoded by nucleotides at the 3′ end of exon 9 and a second continuous region of 2 to 8 amino acids that is encoded by nucleotides at the 5′ end of exon 11.

In other embodiments where, for example, CHK1sv2 polypeptides are used to develop antibodies that bind specifically to CHK1sv2 and not to other CHK1 isoforms, the CHK1sv2 polypeptides comprise at least 10 amino acids of the CHK1sv2 polypeptide sequence corresponding to a junction polynucleotide region created by the alternative splicing of exon 2 to exon 4 of the primary transcript of the CHK1 gene (see FIG. 1). For example, the amino acid sequence: amino terminus-KVPMEKPDIG-carboxy terminus (SEQ ID NO 10), represents one embodiment of such an inventive CHK1sv2 polypeptide wherein a first 5 amino acid region is encoded by a nucleotide sequence at the 3′ end of exon 2 of the CHK1 gene, the 6^{th} amino acid is encoded by nucleotide sequence at the exon 2/exon 4 splice junction, and a carboxy terminal 4 amino acid region is encoded by a nucleotide sequence at the 5′ end of exon 4 of the CHK1 gene. Preferably, at least 10 amino acids of the CHK1sv1 polypeptide comprises a first continuous region of 2 to 8 amino acids that is encoded by nucleotides at the 3′ end of exon 2 and a second continuous region of 2 to 8 amino acids that is encoded by nucleotides at the 5′ end of exon 4.

In other embodiments, CHK1sv1-specific antibodies are made using a CHK1sv1 polypeptide that comprises at least 20, 30, 40 or 50 amino acids of the CHK1sv1 sequence that corresponds to a junction polynucleotide region created by the alternative splicing of exon 9 to exon 11 of the primary transcript of the CHK1 gene. In each case the CHK1sv1 polypeptides are selected to comprise a first continuous region of at least 5 to 15 amino acids that is encoded by nucleotides at the 3′ end of exon 9 and a second continuous region of 5 to 15 amino acids that is encoded by nucleotides directly after the novel splice junction.

In other embodiments, CHK1sv2-specific antibodies are made using an CHK1sv2 polypeptide that comprises at least 20, 30, 40 or 50 amino acids of the CHK1sv2 sequence that corresponds to a junction polynucleotide region created by the alternative splicing of exon 2 to exon 4 of the primary transcript of the CHK1 gene. In each case the CHK1sv2 polypeptides are selected to comprise a first continuous region of at least 5 to 15 amino acids that is encoded by nucleotides at the 3′ end of exon 2 and a second continuous region of 5 to 15 amino acids that is encoded by nucleotides directly after the novel splice junction.

Antibodies to CHK1sv1 or CHK1sv2 have different uses, such as to identify the presence of CHK1sv1 or CHK1sv2, respectively, and to isolate CHK1sv1 or CHK1sv2 polypeptides, respectively. Identifying the presence of CHK1sv1 can be used, for example, to identify cells producing CHK1sv1. Such identification provides an additional source of CHK1sv1 and can be used to distinguish cells known to produce CHK1sv1 from cells that do not produce CHK1sv1. For example, antibodies to CHK1sv1 can distinguish human cells expressing CHK1sv1 from human cells not expressing CHK1sv1 or non-human cells (including bacteria) that do not express CHK1sv1. Such CHK1sv1 antibodies can also be used to determine the effectiveness of CHK1sv1 ligands, using techniques well known in the art, to detect and quantify changes in the protein levels of CHK1sv1 in cellular extracts, and in situ immunostaining of cells and tissues. In addition, the same above-described utilities also exist for CHK1sv2-specific antibodies.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

CHK1sv1 and CHK1sv2 Binding Assay

A number of compounds known to modulate CHK1 activity have been disclosed. Indolocarbazoles such as UCN-01, Gö6976, SB-218078, and ICP-1 act as inhibitors of CHK1 function (Busby et al., 2000, Cancer Res. 60:2108-2112; Kohn et al., 2003, Cancer Res. 63:31-35; Jackson et al., 2000, 60:566-572; Eastman et al., 2002, Mol. Cancer Ther. 1:1067-1078). In addition, certain diaryl ureas and phenyl ureas that effectively inhibit CHK1 have been described (U.S. Patent Application 2004/0014765). Methods for screening compounds for their effects on CHK1 activity have also been disclosed (see for example U.S. Patent Applications 2003/0166216 and 2003/0235899). A person skilled in the art may use these methods to screen CHK1sv1 or CHK1sv2 polypeptides for compounds that bind to, and in some cases functionally alter, each respective CHK1 isoform protein.

CHK1sv1, CHK1sv2, or fragments thereof, can be used in binding studies to identify compounds binding to or interacting with CHK1sv1, CHK1sv2, or fragments thereof, respectively. In one embodiment, CHK1sv1, or a fragment thereof, can be used in binding studies with CHK1 isoform protein, or a fragment thereof, to identify compounds that: bind to or interact with CHK1sv1 and other CHK1 isoforms; bind to or interact with one or more other CHK1 isoforms and not with CHK1sv1. A similar series of compound screens can also be performed using CHK1sv2 rather than, or in addition to, CHK1sv1. Such binding studies can be performed using different formats including competitive and non-competitive formats. Further competition studies can be carried out using additional compounds determined to bind to CHK1sv1, CHK1sv2, or other CHK1 isoforms.

The particular CHK1sv1 or CHK1sv2 sequence involved in ligand binding can be identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using CHK1sv1 expressed from a recombinant nucleic acid. Alternatively, recombinantly expressed CHK1sv1 consists of the SEQ ID NO 4 amino acid sequence. In addition, binding studies are performed using CHK1sv2 expressed from a recombinant nucleic acid. Alternatively, recombinantly expressed CHK1sv2 consists of the SEQ ID NO 6 amino acid sequence.

Binding assays can be performed using individual compounds or preparations containing different numbers of compounds. A preparation containing different numbers of compounds having the ability to bind to CHK1sv1 or CHK1sv2 can be divided into smaller groups of compounds that can be tested to identify the compound(s) binding to CHK1sv1 or CHK1sv2, respectively.

Binding assays can be performed using recombinantly produced CHK1sv1 or CHK1sv2 present in different environments. Such environments include, for example, cell extracts and purified cell extracts containing a CHK1sv1 or CHK1sv2 recombinant nucleic acid; and also include, for example, the use of a purified CHK1sv1 or CHK1sv2 polypeptide produced by recombinant means which is introduced into different environments.

In one embodiment of the invention, a binding method is provided for screening for a compound able to bind selectively to CHK1sv1. The method comprises the steps: providing a CHK1sv1 polypeptide comprising SEQ ID NO 4; providing a CHK1 isoform polypeptide that is not CHK1sv1; contacting the CHK1sv1 polypeptide and the CHK1 isoform polypeptide that is not CHK1sv1 with a test preparation comprising one or more test compounds; and then determining the binding of the test preparation to the CHK1sv1 polypeptide and to the CHK1 isoform polypeptide that is not CHK1sv1, wherein a test preparation that binds to the CHK1sv1 polypeptide, but does not bind to CHK1 isoform polypeptide that is not CHK1sv1, contains one or more compounds that selectively bind to CHK1sv1.

In another embodiment of the invention, a binding method is provided for screening for a compound able to bind selectively to CHK1sv2. The method comprises the steps: providing a CHK1sv2 polypeptide comprising SEQ ID NO 6; providing a CHK1 isoform polypeptide that is not CHK1sv2; contacting the CHK1sv2 polypeptide and the CHK1 isoform polypeptide that is not CHK1sv2 with a test preparation comprising one or more test compounds; and then determining the binding of the test preparation to the CHK1sv2 polypeptide and to the CHK1 isoform polypeptide that is not CHK1sv2, wherein a test preparation that binds to the CHK1sv2 polypeptide, but does not bind to CHK1 isoform polypeptide that is not CHK1sv2, contains one or more compounds that selectively binds to CHK1sv2.

In another embodiment of the invention, a binding method is provided for screening a compound able to inhibit or alter the interaction between CHK1sv1 and CHK1 ligands as a means for identifying modulators of CHK1sv1. As used herein, a CHK1 ligand is any molecule that specifically binds to CHK1, including but not limited to small molecules, macromolecules, peptides, proteins, and protein complexes. Examples of CHK1 ligands include, but are not limited to, CDC25, ATP, TLK1/2, and p53. As used herein, CDC25 is any isoform of any CDC25 phosphatase from any organism, including, but not limited to, human CDC25A, CDC25B, and CDC25C. CHK1 has been shown to specifically bind to and phosphorylate CDC25 dual specificity phosphatases (Sanchez et al, 1997, Science 277:1497-1501; Peng et al., 1997, Science 277:1501-1505; Furnari et al., 1997, Science 277:1495-1497; Mailand et al., 2000, Science 288:1425-1429; Zhao et al., 2002, Proc. Natl. Acad. Sci. USA 99:14795-14800). The method comprises the steps: providing a CHK1sv1 polypeptide comprising SEQ ID NO 4; providing to said CHK1sv1 polypeptide a CHK1 ligand and a test preparation comprising one or more test compounds; and then determining the binding of CHK1 ligand to CHK1sv1 polypeptide, wherein a test preparation that alters the binding of CHK ligand to CHK1sv1 polypeptide contains a compound that binds to or interacts with CHK1sv1 or CHK1 ligand. Alternatively, the above method can be used to identify compounds that inhibit or alter the interaction between CHK1sv2 and CHK1 ligands by performing the method with CHK1sv2 protein comprising SEQ ID NO 6. The identified test compounds may also be tested using the CHK1sv1 or CHK1sv2 functional assays described below.

In another embodiment of the invention, a binding method is provided for screening for a compound able to bind selectively to a CHK1 isoform polypeptide that is not CHK1sv1. The method comprises the steps: providing a CHK1sv1 polypeptide comprising SEQ ID NO 4; providing a CHK1 isoform polypeptide that is not CHK1sv1; contacting the CHK1sv1 polypeptide and the CHK1 isoform polypeptide that is not CHK1sv1 with a test preparation comprising one or more test compounds; and then determining the binding of the test preparation to the CHK1sv1 polypeptide and the CHK1 isoform polypeptide that is not CHK1sv1, wherein a test preparation that binds the CHK1 isoform polypeptide that is not CHK1sv1, but does not bind CHK1sv1, contains a compound that selectively binds the CHK1 isoform polypeptide that is not CHK1sv1. Alternatively, the above method can be used to identify compounds that bind selectively to a CHK1 isoform polypeptide that is not CHK1sv2 by performing the method with CHK1sv2 protein comprising SEQ ID NO 6.

The above-described selective binding assays can also be performed with a polypeptide fragment of CHK1sv1 or CHK1sv2, wherein the polypeptide fragment comprises at least 10 consecutive amino acids that are coded by a nucleotide sequence that bridges the junction created by the splicing of the 3′ end of exon 9 to the 5′ end of exon 11 in the case of CHK1sv1 or by the splicing of the 3′ end of exon 2 to the 5′ end of exon 4, in the case of CHK1sv2. Similarly, the selective binding assays may also be performed using a polypeptide fragment of an CHK1 isoform polypeptide that is not CHK1sv1 or CHK1sv2, wherein the polypeptide fragment comprises at least 10 consecutive amino acids that are coded by: a) a nucleotide sequence that is contained within exons 10 or 3, of the CHK1 gene; or b) a nucleotide sequence that bridges the junction created by the splicing of the 3′ end of exon 9 to the 5′ end of exon 10, the splicing of the 3′ end of exon 10 to the 5′ end of exon 11, the splicing of the 3′ end of exon 2 to the 5′ end of exon 3, or the splicing of the 3′ end of exon 3 to the 5′ end of exon 4 of the CHK1 gene.

CHK1 Functional Assays

CHK1 encodes checkpoint kinase 1, a component of the DNA damage response pathway that plays an integral role in the cascade leading to the inactivation of CDC25 and the transcription of genes in response to DNA damage. The identification of CHK1sv1 and CHK1sv2 as splice variants of CHK1 provides a means for screening for compounds that bind to CHK1sv1 and/or CHK1sv2 protein thereby altering the activity or regulation of CHK1sv1 and/or CHK1sv2 polypeptides. Assays involving a functional CHK1sv1 or CHK1sv2 polypeptide can be employed for different purposes, such as selecting for compounds active at CHK1sv1 or CHK1sv2; evaluating the ability of a compound to affect the kinase activity of each respective splice variant polypeptide; and mapping the activity of different CHK1sv1 and CHK1sv2 regions. CHK1sv1 and CHK1sv2 activity can be measured using different techniques such as: detecting a change in the intracellular conformation of CHK1sv1 or CHK1sv2; detecting a change in the intracellular location of CHK1sv1 or CHK1sv2; or by measuring the kinase activity of CHK1sv1 or CHK1sv2.

In one embodiment of the invention, a method is provided for screening a compound able to modulate the kinase activity of CHK1sv1. The method comprises the steps: providing a CHK1sv1 polypeptide comprising SEQ ID NO 4; providing to said CHK1sv1 polypeptide a kinase substrates and a test preparation comprising one or more test compounds; and then determining the kinase activity of said CHK1sv1 polypeptide, wherein a test preparation that alters the CHK1sv1 activity, compared to the CHK1sv1 activity in the absence of the test preparation, contains a compound that modulates CHK1sv1 kinase activity. Suitable kinase substrates include, for example, natural substrates such as CDC25, TLK1/2, and p53, as well as any other natural or synthetic molecule that is phosphorylated to a measurable extent by a CHK1 isoform polypeptide. Alternatively, the above method can be used to identify compounds that alter the kinase activity of CHK1sv2 by performing the method with CHK1sv2 protein comprising SEQ ID NO 6. The above methods are performed using any kinase assay conditions that permit statistically significant measurement of CHK1 kinase activity, as provided in, but not limited by, the example sections herein, references cited herein, and as would otherwise be apparent to a person of skill in the art.

Recombinantly expressed CHK1sv1 and CHK1sv2 can be used to facilitate the determination of whether a compound's activity in a cell is dependent upon the presence of CHK1sv1 or CHK1sv2. For example, CHK1sv1 can be expressed by an expression vector in a cell line and used in a co-culture growth assay, such as described in U.S. Pat. No. 6,518,035, to identify compounds that alter the growth of the cell expressing CHK1sv1 from the expression vector as compared to the same cell line but lacking the CHK1sv1 expression vector. Alternatively, determination of whether a compound's activity on a cell is dependent upon the presence of CHK1sv1 or CHK1sv2 can also be done using gene expression profile analysis methods as described, for example, in U.S. Pat. No. 6,324,479. Similar assays can also be used for CHK1sv2.

Methods to determine CHK1 activation or its function are well known in the art. Various kinase assays are used to determine the activity of CHK1 (see for example, Chen et al., 2000, Cell, 100:681-692; Zhao et al., 2002, Proc. Natl. Acad. Sci. U.S.A. 99:14795-14800; and Ng et al., 2004, J. Biol. Chem. 279:8808-8819). CHK1 protein can be affinity purified from cellular lysates and then added to kinase reactions consisting of kinase buffer, [γ-³²P]ATP, and soluble CDC25C₂₀₀₋₂₅₆ peptide substrate. Following incubation of reaction mixtures, they can be resolved by SDS-PAGE, and the proteins then transferred to nitrocellulose membrane. Radiolabeled proteins can be visualized by autoradiography, and the same membrane may then be probed with anti-CHK1 monoclonal antibodies (Zhao and Piwnica-Worms, 2001, Mol. Cell Biol. 21:4129-4139). Assays determining abrogation of G₂ arrest and induction of apoptosis in response to DNA damage have also been described as a measure of CHK1 activity (Xiao et al., 2003, J. Biol. Chem. 278:21767-21773; Chen et al., 2003, Mol. Cancer Ther. 2:543-548; Suganuma et al., 1999, Cancer Res. 59:5887-5891). High throughput screening methods for identifying CHK1 kinase domain inhibitors have also been described (US 2003/0235899). A variety of other assays has been used to investigate the properties of CHK1 and therefore would also be applicable to the measurement of CHK1sv1 or CHK1sv2 functions.

CHK1sv1 or CHK1sv2 functional assays can be performed using cells expressing CHK1sv1 or CHK1sv2. These proteins can be contacted with individual compounds or preparations containing different compounds. A preparation containing different compounds where one or more compounds affect CHK1sv1 or CHK1sv2 in cells over-producing CHK1sv1 or CHK1sv2 as compared to control cells containing an expression vector lacking CHK1sv1 or CHK1sv2 coding sequences, can be divided into smaller groups of compounds to identify the compound(s) affecting CHK1sv1 or CHK1sv2 activity, respectively.

CHK1sv1 or CHK1sv2 functional assays can be performed using recombinantly produced CHK1sv1 or CHK1sv2 present in different environments. Such environments include, for example, cell extracts and purified cell extracts containing CHK1sv1 or CHK1sv2 expressed from recombinant nucleic acid; and the use of purified CHK1sv1 or CHK1sv2 produced by recombinant means that is introduced into a different environment suitable for measuring binding or kinase activity.

Modulating CHK1sv1 and CHK1sv2 Expression

CHK1sv1 or CHK1sv2 expression can be modulated as a means for increasing or decreasing CHK1sv1 or CHK1sv2 activity, respectively. Such modulation includes inhibiting the activity of nucleic acids encoding the CHK1 isoform target to reduce CHK1 isoform protein or polypeptide expression, or supplying CHK1 nucleic acids to increase the level of expression of the CHK1 target polypeptide thereby increasing CHK1 activity.

Inhibition of CHK1sv1 and CHK1sv2 Activity

CHK1sv1 or CHK1sv2 nucleic acid activity can be inhibited using nucleic acids recognizing CHK1sv1 or CHK1sv2 nucleic acid and affecting the ability of such nucleic acid to be transcribed or translated. Inhibition of CHK1sv1 or CHK1sv2 nucleic acid activity can be used, for example, in target validation studies.

A preferred target for inhibiting CHK1sv1 or CHK1sv2 is mRNA stability and translation. The ability of CHK1sv1 or CHK1sv2 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNA inhibition (RNAi) using shRNA or siRNA molecules can also be used to prevent protein expression of a target transcript. This method is based on the interfering properties of double-stranded RNA derived from the coding regions of the gene that disrupt the synthesis of protein from transcribed RNA.

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Modified and unmodified nucleic acids can be used as anti-sense molecules, RNAi and ribozymes. Different types of modifications can affect certain anti-sense activities such as the ability to be cleaved by RNAse H, and can alter nucleic acid stability. Examples of references describing different anti-sense molecules, and ribozymes, and the use of such molecules, are provided in U.S. Pat. Nos. 5,849,902; 5,859,221; 5,852,188; and 5,616,459. Methods for modulating target gene transcription by administering enzymatic nucleic acids having a sequence specific for CHK1 sequence have been described (U.S. Patent Application No. 2003/0087847 A1). Techniques for reducing the activity of CHK1 by delivering anti-sense oligodeoxynucleotides specific for an RNA encoding CHK1 have been disclosed (U.S. Pat. No. 6,211,164 B1). Examples of organisms in which RNAi has been used to inhibit expression of a target gene include: C. elegans (Tabara, et al., 1999, Cell 99, 123-32; Fire, et al., 1998, Nature 391, 806-11), plants (Hamilton and Baulcombe, 1999, Science 286, 950-52), Drosophila (Hammond, et al., 2001, Science 293, 1146-50; Misquitta and Patterson, 1999, Proc. Nat. Acad. Sci. 96, 1451-56; Kennerdell and Carthew, 1998, Cell 95, 1017-26), and mammalian cells (Bernstein, et al., 2001, Nature 409, 363-6; Elbashir, et al., 2001, Nature 411, 494-8).

Increasing CHK1sv1 and CHK1sv2 Expression

Nucleic acids encoding CHK1sv1 or CHK1sv2 can be used, for example, to cause an increase in CHK1 activity or to create a test system (e.g., a transgenic animal) for screening for compounds affecting CHK1sv1 or CHK1sv2 expression, respectively. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra Nucleic acid can be introduced into cells present in different environments using in vitro, in vivo, or ex vivo techniques. Examples of techniques useful in gene therapy are illustrated in Gene Therapy &Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of CHK1sv1 Using Real-Time PCR

To identify variants of the “normal” splicing of exon regions encoding CHK1, RT-PCR and real-time PCR assays were used. In particular, splicing variations resulting in the loss of the C-terminal regulatory domain of CHK1 were sought. Deletion of the C-terminus confers greater kinase activity to CHK1 (Chen et al., 2000, Cell 100:681-692; Katsuragi and Sagata, 2004, Mol. Biol. Cell. 15:1680-1689). Exons 2-8 encode the catalytic kinase domain and exon 9 encodes the linker region. The SQ and C-terminal regulatory domains lie within exons 10-13 (Sanchez et al., 1997, 277:1497-1501; Katsuragi and Sagata, 2004, Mol. Biol. Cell. 15:1680-1689).

RT-PCR

The structure of CHK1 mRNA in the region corresponding to exons 8 to 11 was determined for RNA extracted from human testis using an RT-PCR based assay. Human testis was selected as the tissue source for the search for alternative CHK1 transcripts, as alternative splicing is relatively common in this tissue (reviewed in Venables, 2002, Curr. Opin. Genet. Dev. 12:615-619; Eddy, 1998, Semin. Cell. Dev. Biol. 9:451-457). Total RNA isolated from human testis was obtained from BD Biosciences Clontech (Palo Alto, Calif.). RT-PCR primers were selected that were complementary to sequences in exon 8 and exon 11 of the reference exon coding sequences in CHK1 (NM_{—}001274). Based upon the nucleotide sequence of CHK1 mRNA, the CHK1 exon 8 and exon 11 primer set (hereafter CHK1₈₋₁₁ primer set) was expected to amplify a 478 base pair amplicon representing the “reference” CHK1 mRNA region. The CHK1₈₋₁₁ primer set was expected to amplify a 300 base pair amplicon in a transcript that possessed alternative splicing of exon 9 to exon 11. The CHK1 exon 8 forward primer has the sequence: 5′ ATCAGCAAGAATTACCATTCCAGACATC 3′ (SEQ ID NO 11); and the CHK1 exon 11 reverse primer has the sequence: 5′CATACAACTTTTCTTCCATTGATAGCCC 3′ (SEQ ID NO 12).

Twenty-five ng of total RNA from human testis was subjected to a one-step reverse transcription-PCR amplification protocol using the Qiagen, Inc. (Valencia, Calif.), One-Step RT-PCR kit, using the following cycling conditions:
50° C. for 30 minutes;
95° C. for 15 minutes;
35 cycles of:
   94° C. for 30 seconds;
   63.5° C. for 40 seconds;
   72° C. for 50 seconds; then
   72° C. for 10 minutes.

RT-PCR amplification products (amplicons) were size fractionated on a 2% agarose gel. Selected fragments representing 250 to 350 base pair amplicons were manually extracted from the gel and purified with a Qiagen Gel Extraction Kit. The purified amplicon fragments were reamplified with the CHK1₈₋₁₁ primer set, and these amplicons were size fractionated on an agarose gel. Fragments representing 250 to 350 base pair amplicons were manually extracted from the gel and purified with a Qiagen Gel Extraction Kit. The purified amplicon fragments were reamplified with the CHK1₈₋₁₁ primer set once more. Following size fractionation on an agarose gel and manual extraction of the 250 to 350 base pair amplicons, the purified amplicon fragments (Qiagen Gel Extraction Kit) were cloned into an Invitrogen pCR2.1 vector using the reagents and instructions provided with the TOPO TA cloning kit (Invitrogen, Carlsbad, Calif.). Clones were then plated in pools of 440 colonies per plate, onto 15 plates, for a total of 6600 clones. DNA was extracted from the pooled 440 colonies from each plate and used as template for real-time PCR.

Real-Time PCR/TAQman

To determine the presence of an alternatively spliced isoform to the CHK1 reference protein (NP_{—}001265), a real-time PCR assay was used.

TAQman primers and probes used to detect the CHK1sv1 isoform were designed and synthesized as pre-set mixtures (Applied Biosystems, Foster City, Calif.). The sequences of the TAQman primers and probes used to detect the CHK1 reference form (SEQ ID NOs 13, 14, and 15) and CHK1sv1 isoform (SEQ ID NOs 16, 17, and 18) are shown in Table 1. Splice junction specific probes were labeled with the 6-FAM fluorophore at the 5′ end (FAM) and a non-fluorescent quencher at the 3′ end (NFQ). Real-time PCR was performed on human testis cDNA using the TaqMan Universal PCR Master Mix (Applied Biosystems, Foster City, Calif.). The TAQman reaction contained:

| 96-well format | 384-well format | |
|---|---|---|
| 12.5 μl | 5 μl | TAQman Universal MasterMix |
| 1.25 μl | 0.5 μl | Primer-probe mix |
| 6.25 μl | 2.5 μl | H₂O |
| 5 μl | 2 μl | DNA |

TABLE 1
Primers and probes used to detect CHK1 isoforms.

| | | | |
|---|---|---|---|
| Name | SEQ ID NO | Sequence | Specificity |

| | | | |
|---|---|---|---|
| CHK1 reference forward | SEQ ID NO 13 | GTTACTTGGCACCCCAGGA | CHK1 reference |
| primer | | | |
| CHK1 reference reverse | SEQ ID NO 14 | CATCCAATTTGGTAAAGAATCGTGTCA | CHK1 reference |
| primer | | | |
| CHK1 reference probe | SEQ ID NO 15 | FAM-TCCTCACAGAACCCC-NFQ | CHK1 reference |
| CHK1sv1 forward primer | SEQ ID NO 16 | GCACATTCAATCCAATTTGGACTTCT | CHK1sv1 |
| CHK1sv1 reverse primer | SEQ ID NO 17 | CATCCAATTTGGTAAAGAATCGTGTCAT | CHK1sv1 |
| CHK1sv1 probe | SEQ ID NO 18 | FAM-CAGTGCTTCTAGAACCC-NFQ | CHK1sv1 |

The TAQman reactions were performed on an ABI Prism 7900HT Sequence Detection System (Applied Biosystems, Foster City, Calif.). The thermocycling conditions were 50° C. for 2 minutes, 95° C. for 10 minutes, and 40 cycles of 95° C. for 15 seconds and 60° C. for 1 minute. Data analysis of the fluorescence emission was performed by the Sequence Detector Software (SDS) (Applied Biosystems, Foster City, Calif.). Briefly, an amplification plot was generated for each sample, which showed cycle number on the x axis vs. ΔRₙ on the y axis. Rn is the fluorescence emission intensity of the reporter dye normalized to a passive reference, and ΔRₙ is the Rₙ value of the reaction minus the Rₙ of an un-reacted sample. A threshold cycle (CT) value, the cycle at which a statistically significant increase in ΔRₙ is first detected, was calculated from the amplification plot. The threshold was automatically calculated by the SDS as the 10-fold standard deviation of the Rₙ in the first 15 cycles. The obtained C_{T} values were exported Microsoft Excel for analysis as recommended by the manufacturer (Applied Biosystems, Foster City, Calif.).

Results of the TAQman assay indicated that .pooled DNA from 13 out of 15 plates appeared to possess clones that represented an alternative exon 9 to exon 11 splice junction. DNA from one of these positive pools, representing 440 colonies, was used to transform bacterial host cells. Clones were plated in pools of 55 colonies per plate onto 12 plates total. The colonies on each of the 12 plates were again pooled and used for a TAQman assay. Pooled DNA from 1 out of 12 plates appeared to possess a clone that represented an alternative exon 9 to exon 11 splice junction. The 55 colonies on this positive plate were individually screened using a TAQman assay, and one clone was identified as possessing an alternative exon 9 to exon 11 splice junction. This positive clone was then sequenced from each end using the CHK1 exon 8 forward primer (SEQ ID NO 11) and a different exon 11 reverse primer with the sequence 5′ TGCATCCAATTTGGTAAAGAATCG 3′ (SEQ ID NO 19) by Qiagen Genomics, Inc. (Bothell, Washington).

Sequence analysis of the clone revealed that it matched the expected sequence for alternative splicing of exon 9 of the CHK1 heteronuclear RNA to exon 11; that is the coding sequence of exon 10 is completely absent.

### Example 2

### Identification of CHK1sv2 Using RT-PCR

The structure of CHK1 mRNA in the region corresponding to exons 2 to 11 was determined for MOLT-4 and Daudi Burkitts lymphoma cell line samples using an RT-PCR based assay. PolyA purified mRNA isolated from MOLT-4 and Daudi Burkitts lymphoma cell line samples were obtained from BD Biosciences Clontech (Palo Alto, Calif.). RT-PCR primers were selected that were complementary to sequences in exon 2 and exon 11 of the reference exon coding sequences in CHK1 (NM_{—}001274). Based upon the nucleotide sequence of CHK1 mRNA, the CHK1 exon 2 and exon 11 primer set (hereafter CHK1₂₋₁₁ primer set) was expected to amplify a 1163 base pair amplicon representing the “reference” CHK1 mRNA region. The CHK1 exon 2 forward primer, has the sequence: 5′ GAGTCATGGCAGTGCCCTTTGT 3′ (SEQ ID NO 20); and the CHK1 exon 11 reverse primer, has the sequence: 5′ TGCATCCAAT TTGGTAAAGAATCG 3′ (SEQ ID NO 19).

Twenty-five ng of polyA mRNA from each cell line was subjected to a one-step reverse transcription-PCR amplification protocol using the Qiagen, Inc. (Valencia, Calif.), One-Step RT-PCR kit, using the following cycling conditions:
50° C. for 30 minutes;
95° C. for 15 minutes;
35 cycles of:
   94° C. for 30 seconds;
   63.5° C. for 40 seconds;
   72° C. for 50 seconds; then
   72° C. for 10 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were sequenced from each end (using the same primers used for RT-PCR) by Qiagen Genomics, Inc. (Bothell, Washington).

At least two different RT-PCR amplicons were obtained from human mRNA samples using the CHK1₂₋₁₁ primer set (data not shown). The MOLT-4 and Daudi cell lines assayed exhibited the expected amplicon size of 1163 base pairs for normally spliced CHK1 mRNA. However, in addition to the expected CHK1 amplicon of 1163 base pairs, MOLT-4 and Daudi cell lines also exhibited an amplicon of about 939 base pairs

Sequence analysis of the about 939 base pair amplicon amplified using the CHK1₂₋₁₁ primer set revealed that this amplicon form results from the splicing of exon 2 of the CHK1 hnRNA to exon 4; that is, exon 3 coding sequence is completely absent. Thus, the RT-PCR results also suggested that CHK1 mRNA is composed of a mixed population of molecules wherein in at least one of the CHK1 mRNA splice junctions is altered.

### Example 3

### Cloning of CHK1sv1 and CHK1sv2

Real-time PCR, RT-PCR, and sequencing data indicate that in addition to the normal CHK1 reference mRNA sequence, NM_{—}001274, encoding CHK1 protein, NP_{—}001265, two novel splice variant forms of CHK1 mRNA also exist in testis tissue and MOLT-4, and Daudi cell lines.

Clones having a nucleotide sequence comprising the CHK1sv1 splice variant identified in Example 1 were isolated using recombination-mediated plasmid construction in yeast. A set of two primer pairs was used to amplify and clone the entire mRNA coding sequences of CHK1sv1. In the case of CHK1sv1, real-time quantitative PCR analysis indicated that transcripts of this splice variant form were present at very low levels (data not shown). In order to clone CHK1sv1, clones containing coding sequences of the reference CHK1 (NM_{—}001274) were altered by an additional recombination step in yeast with 80 base pair linkers that were designed to create the desired exon 9 to exon 11 splice junction.

A 5′ “forward” primer and a 3′ “reverse” primer were designed for isolation of full length clones corresponding to CHK1sv1. The 5′ “forward” CHK1sv1 primer was designed to have the nucleotide sequence of 5′ TTACTGGCTTATCGAAATTAATACGACTCACTATAG GGAGGAGTCATGGCAGTGCCCTTTGT 3′ (SEQ ID NO 21) and to have sequences complementary to exon 2 of the CHK1 mRNA (NM_{—}001274). The 3′ “reverse” CHK1sv1 primer was designed to have the nucleotide sequence of 5′ TAGAAGGCACAGTCGAGGCTGA TCAGCGGGTTTAAACTCATGCATCCAATTTGGTAAAGAATCG 3′ (SEQ ID NO 22) and to have sequences complementary to exon 11 of the CHK1 mRNA (NM_{—}001274). The 40 nucleotides at the 5′ ends of the primer sequences indicated in italics are “tails” that were incorporated into the PCR amplicons and facilitated subsequent plasmid recombination events in yeast. These CHK1sv1 “forward” and “reverse” primers were expected to amplify coding sequences of the reference CHK1 mRNA (NM_{—}001274), which was then used in a subsequent recombinational cloning step to create CHK1sv1-specific sequence.

RT-PCR

The CHK1sv1 cDNA sequence was cloned using a combination of reverse transcription (RT) and polymerase chain reaction (PCR). More specifically, about 25 ng of MOLT-4 cell line mRNA (BD Biosciences Clontech, Palo alto, CA) was reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction was added to 40 μl of water, 5 μl of 10× buffer, 1 μl of dNTPs and 1 μl of enzyme from a Clontech (Palo Alto, Calif.) Advantage 2 PCR kit. PCR was done in a Gene Amp PCR System 9700 (Applied Biosystems, Foster City, Calif.) using the CHK1sv1 “forward” and “reverse” primers for CHK1sv1 (SEQ ID NOs 21-22). After an initial 94° C. denaturation of 1 minute, 35 cycles of amplification were performed using a 30 second denaturation at 94° C. followed by a 40 second annealing at 63.5° C. and a 50 second synthesis at 72° C. The 35 cycles of PCR were followed by a 10 minute extension at 72° C. The 50 μl reaction was then chilled to 4° C. 10 μl of the resulting reaction product was run on a 1% agarose (Invitrogen, Ultra pure) gel stained with 0.3 μg/ml ethidium bromide (Fisher Biotech, Fair Lawn, N.J.). Nucleic acid bands in the gel were visualized and photographed on a UV light box to determine if the PCR had yielded products of the expected size, in the case of the CHK1 mRNA, a product of about 1243 base pairs. The remainder of the 50 μl PCR reactions from MOLT-4 cells was purified using the QIAquik Gel extraction Kit (Qiagen, Valencia, Calif.) following the QIAquik PCR Purification Protocol provided with the kit. About 50 μl of product obtained from the purification protocol was concentrated to about 6 μl by drying in a Speed Vac Plus (SC110A, from Savant, Holbrook, N.Y.) attached to a Universal Vacuum System 400 (also from Savant) for about 30 minutes on medium heat.

Construction of Cycloheximide-Resistant Saccharomyces cerevisiae Strain

A cycloheximide-based counterselection was used to increase specificity of cloning by homologous recombination relative to nonspecific vector background (Raymond et al., 2002, Genome Res. 12:190-197). Plasmid re-circularization is the primary source of background in recombinational cloning experiments (Boulton and Jackson, 1996, Nucleic Acids Res. 24:4639-4648; Raymond et al., 1999, Biotechniques 26:134-8, 140-1). The yeast strain used in this study, CMY1-5, is cycloheximide resistant (CYH2^{R}). The introduction of a vector with the wild-type CYH2 allele into this yeast strain confers dominant sensitivity to cycloheximide. The position of the CYH2 gene in the cloning vector relative to the targeted recombination sites was designed such that the CYH2 gene was lost in recombinant clones but retained in most end-joined plasmids. Therefore, yeast cells containing recombinant plasmids were selected in the presence of cycloheximide, while yeast cells containing non-recombinant plasmids were sensitive to the drug. However, any marker that confers dominant sensitivity could be used in a counterselection experiment, such as URA3, 5-fluoroorotic acid, LYS2 and α-amino adipic acid, or CAN1.

A cycloheximide resistant strain was generated from the cycloheximide sensitive yeast strain BY4709 (Brachmann et al, 1998, Yeast 14:115-132). Two overlapping 1 kb products from the CYH2 gene were amplified from BY4709 yeast strain using two sets of tailed primers (hereafter Set 1 and Set 2) listed in Table 2, which introduce a critical Q→E amino acid change at residue 38 in the CYH2 protein, thereby conferring drug resistance. The 5′ “forward” Set 1 primer was designed to have the nucleotide sequence of 5′ ATACGACTCACTATAGGG AGACCCAAGCTGGCTAGTTAAGTATGTTTATATATGGATTTTGAAA 3′ (SEQ ID NO 23). The 3′ “reverse” Set 1 primer was designed to have the nucleotide sequence of 5′ GTA GAGGTATGGCCGGTGGTGAACATCACCACAGAATTAAC 3′ (SEQ ID NO 24). The 5′ “forward” Set 2 primer was designed to have the nucleotide sequence of 5′ TCCACCACACTG GACTAGTGGATCCGAGCTCGGTACCAAGGCCCGGGCATGCTACGTACCTGTTTAACT CTTC 3′ (SEQ ID NO 25). The 3′ “reverse” Set 2 primer was designed to have the nucleotide sequence of 5′ GTTAATTCTGTGGTGATGTTCACCACCGGCCATACCTCTAC 3′ (SEQ ID NO 26).

TABLE 2

Tailed PCR primer sets used to amplify CYH2
and introduce a drug-resistant mutation

| | | |
|---|---|---|
| Primer Name | SEQ ID NO | Primer Sequence |

| | | |
|---|---|---|
| Set 1 Forward | SEQ ID NO 23 | ATACGACTCACTATAGGGAGACCCAAGCTGGCTAGTTAAGTATGTTTATAT |
| | | ATGGATTTTGAAA |
| Set 1 Reverse | SEQ ID NO 24 | GTAGAGGTATGGCCGGTGGTGAACATCACCACAGAATTAAC |
| Set 2 Forward | SEQ ID NO 25 | TCCACCACACTGGACTAGTGGATCCGAGCTGGGTACCAAGGCCCGGGCAT |
| | | GCTACGTACCTGTTTAACTCTTC |
| Set 2 Reverse | SEQ ID NO 26 | GTTAATTCTGTGGTGATGTTCACCACCGGCCATACCTCTAC |

Recombination of the Set 1 and Set 2 CYH2 amplicons into the endogenous yeast CYH2 gene confers cycloheximide resistance. 1 μg each of the Set 1 and Set 2 PCR amplicons, which incorporate the Q38E mutation, were cotransformed by electroporation as described in Raymond et al. (2002, Genome Res. 12:190-197) with yeast strain BY4709 (Brachmann et al., 1998 Yeast, 14:115-132), and cycloheximide resistant colonies were selected on media containing 1 μg/ml cycloheximide (Sigma, St. Louis, Mo.). Yeast transformation methods, including electroporation, lithium acetate treatment, and spheroplasting, are also discussed in Gietz and Woods (2001 Biotechniques, 30:816-820, 822-826, 828). One cycloheximide resistant strain, CMY1-5 (Matα, ura3Δ, cyh2^{R}) was used for all subsequent studies.

Construction of Yeast Plasmids

Plasmids that are assembled in yeast by recombination typically include sequence elements that allow their selection in yeast (e.g., CYH2 or URA3 resistance). Sequence elements that permit plasmid replication include a yeast centromere sequence and yeast DNA autonomously replicating sequence. DNA sequences for selection (e.g., ampicillin, kanamycin, or chloramphenicol resistance) and replication (e.g., colE1 or mini F′) in Escherichia coli are typically included in the plasmid to allow transformation of E. coli for the preparation of large quantities of recombinant plasmid. Isolation of yeast plasmid for bacterial transformation is described in Hoffman and Winston (1987, Gene 57:267-72). One such embodiment of a yeast plasmid is the sequences found in the vector pRS316 (Sikorski and Hieter, 1989, Genetics 122:19-27). Additional elements including promoters, terminators, and selectable markers for recombinant expression in mammalian cells can be found in commercially available plasmid vectors and incorporated into yeast cloning vectors.

Overlapping sequences are used to target recombinational cloning of DNA fragments into specific sites in the target yeast plasmid. The region of overlap can be as short as 20 bp, but is optimally 40 bp or longer (Hudson et al, 1997, Genome Res. 7:1169-1173; Oldenburg et al., 1997 Nucleic Acids Res. 25:451-452; Hua et al., 1997, Plasmid 38:91-96; Raymond et al, 1999, Biotechniques, 26:134-8, 140-1). Sequence homology for these overlapping regions may be provided by amplification of target sequences with PCR primers that include varying lengths of base pair extensions (“tails”) that become incorporated into the amplicons. Alternatively, synthetic oligonucleotide recombination linkers that provide sequence homology at each end to the two unrelated DNA molecules to be joined may be used (Raymond et al, 2002, Genome Res. 12:190-197; DeMarini et al., 2001, Biotechniques 30:520-523).

To create the cloning vectors used for this study, plasmid pRS416 (ATCC No. 87521) (Sikorski and Hieter, 1989, Genetics, 122:19-27) was digested with SspI; and plasmid pENTR11 (InVitrogen, Carlsbad, Calif.) was digested with NheI. BY4709 yeast strain was cotransformed with the two plasmid vector fragments and the recombinational linkers listed in Table 3 (SEQ ID NOs 27-30) by electroporation (Raymond et al., 2002 Genome Res. 12:190-197) to form the resulting pCMR2 plasmid (SEQ ID NO 31). All subsequent transformation steps for plasmid construction were performed with yeast strain BY4709 using the referenced electroporation method unless otherwise indicated.

TABLE 3
Linkers used to joining pRS416 (SspI) and pENTR11 (NheI).

| | |
|---|---|
| SEQ ID NO | Linker Sequence |

| | |
|---|---|
| SEQ ID NO 27 | TGCGTTATCCCCTGATTCTGTGGATAACCGTATTACCGCTGGGTAATAACTGATATAATTAAAT |
| | TGAAGCTCTAATTTGT |
| SEQ ID NO 28 | ACAAATTAGAGCTTCAATTTAATTATATCAGTTATTACCCAGCGGTAATACGGTTATCCACAGA |
| | ATCAGGGGATAACGCA |
| SEQ ID NO 29 | AATTTAAATTATAATTATTTTTATAGCACGTGATGAAAAGAGCATGGATCTCGGGGACGTCTA |
| | ACTACTAAGCGAGAGTA |
| SEQ ID NO 30 | TACTCTCGCTTAGTAGTTAGACGTCCCCGAGATCCATGCTCTTTTCATCACGTGCTATAAAAAT |
| | AATTATAATTTAAATT |

pCMR2 plasmid (SEQ ID NO 31) was cut with SmaI and then recombined with the linkers in Table 4 (SEQ ID NO 32, 33) to produce pCMR3 (SEQ ID NO 34).

TABLE 4
Linkers used to convert pCMR2 to pCMR3.

| | |
|---|---|
| SEQ ID NO | Linker Sequence |

| | |
|---|---|
| SEQ ID NO 32 | ACTTTGTACAAAAAAGCAGGCTTCGAAGGAGATAGAACCAGCCCGGGCGCCGCACTCGAGATA |
| | TCTAGACCCAGCTTTCTTGTACAAA |
| SEQ ID NO 33 | |
| TTTGTACAAGAAAGCTGGGTCTAGATATCTCGAGTGCGGCGCCCGGGCTGGTTCTATCTCCTTC | |
| | GAAGCCTGCTTTTTTGTACAAAGT |

pCMR3 (SEQ ID NO 34) was digested with SspI, and pCCFOS1 (EpiCentre Technologies, Madison, Wis.) was cut with SalI. The vector fragments were then joined with the linkers shown in Table 5 (SEQ ID NOs 35-38), resulting in plasmid pCMR7 (SEQ ID NO 39).

TABLE 5
Linkers used to join pCMR3 and pCCFOS1.

| | |
|---|---|
| SEQ ID NO | Linker Sequence |

| | |
|---|---|
| SEQ ID NO 35 | GTTAACCGGGCTGCATCCGATGCAAGTGTGTCGCTGTCGAGGGTAATAACTGATATAATTAAA |
| | TTGAAGCTCTAATTTGT |
| SEQ ID NO 36 | ACAAATTAGAGCTTCAATTTAATTATATCAGTTATTACCCTCGACAGCGACACACTTGCATCGG |
| | ATGCAGCCCGGTTAAC |
| SEQ ID NO 37 | ATAAAATCATTATTTGCCATCCAGCTGCAGCTCTGGCCCGTCGAATTTCTGCCATTCATCCGCT |
| | TATTATCACTTATTCA |
| SEQ ID NO 38 | TGAATAAGTGATAATAAGCGGATGAATGGCAGAAATTCGACGGGCCAGAGCTGCAGCTGGAT |
| | GGCAAATAATGATTTTAT |

Plasmid pCMR7 (SEQ ID NO 39) was cut with SrfI; plasmid pcDNA3.1 mycHIS A (InVitrogen) was cut with SspI. The resulting plasmid fragments were joined with the linkers shown in Table 6 (SEQ ID NOs 40-43), yielding plasmid pCMR9 (SEQ ID NO 44).

TABLE 6
Linkers used to join pCMR7 and pcDNA3.1 mycHIS A.

| | |
|---|---|
| SEQ ID NO | Linker Sequence |

| | |
|---|---|
| SEQ ID NO 40 | ACGGTTATCCACAGAATCAGGGGATAACGCAGGAAAGAACATAAAATCATTATTTGCCATCCA |
| | GCTGCAGCTCTGGCCCG |
| SEQ ID NO 41 | CGGGCCAGAGCTGCAGCTGGATGGCAAATAATGATTTTATGTTCTTTCCTGCGTTATCCCCTGA |
| | TTCTGTGGATAACCGT |
| SEQ ID NO 42 | AATTTAAATTATAATTATTTTTATAGCACGTGATGAAAAGTCCGCGCACATTTCCCCGAAAAGT |
| | GCCACCTGACGTCGAC |
| SEQ ID NO 43 | GTCGACGTCAGGTGGCACTTTTCGGGGAAATGTGCGCGGACTTTTCATCACGTGCTATAAAAA |
| | TAATTATAATTTAAATT |

Construct pCMR9 (SEQ ID NO 44) was cut with HindIII. The yeast CYH2 gene was amplified as two 1 kb overlapping pieces from yeast strain BY4709 (Brachmann et al, 1998, Yeast 14:155-132), using the two sets of tailed primers listed in Table 7. The sequences of one set of CYH2 primers are set forth in SEQ ID NOs 23 and 45; the sequences of the second set of primers are set forth in SEQ ID NOs 25 and 46. The full length CYH2 gene was assembled into pCMR9 by cotransformation of CMY1-5 yeast strain to produce pCMR11 (SEQ ID NO 47) (see Table 8).

TABLE 7
Tailed PCR primers used to amplify CYH2 for recombination into pCMR9.

| | |
|---|---|
| SEQ ID NO | Primer Sequence |

| | |
|---|---|
| SEQ ID NO 23 | ATACGACTCACTATAGGGAGACCCAAGCTGGCTAGTTAAGTATGTTTATATATGGATTTTGAAA |
| SEQ ID NO 45 | GTAGAGGTATGGCCGGTGGTCAACATCACCACAGAATTAAC |
| SEQ ID NO 25 | TCCACCACACTGGACTAGTGGATCCGAGCTCGGTACCAAGGCCCGGGCATGCTACGTACCTG |
| | TTTAACTCTTC |
| SEQ ID NO 46 | GTTAATTCTGTGGTGATGTTGACCACCGGCCATACCTCTAC |

TABLE 8
Composition of pCMR10 and pCMR11 plasmids

| Nucleotide | |
|---|---|
| coordinates | Functional description of sequence |
| 1-6013 | Copy-control ™ E. coli origin |
| | of replication from pCC1FOS (Epicentre |
| | Technologies, Madison, WI). |
| 6014-7884 | Yeast URA3 gene, ARS4 |
| | autonomously replicating sequence and CEN6 |
| | centromere from pRS316 (Sikorski and Hieter, 1989). |
| 7885-8825 | Mammalian CMV promoter from InVitrogen (Carlsbad, |
| | CA) vector pcDNA3.1/myc-HIS A. |
| 8826-10,774 | Yeast CYH2 gene amplified from strain |
| | BY4709 (Brachmann et al. 1998) |
| 10,775-10,782 | Engineered SrfI restriction site. |
| 10,783-13,556 | Mammalian poly-adenylation sites, selectable |
| | markers, SV40 origin, etc. from pcDNA3.1/myc-HIS A. |
| 13,557-13,596 | DNA sequence from InVitrogen vector pENTR11. |
| 13,597-14,567 | pCMR10 - specific; kanamycin resistance gene |
| | from pENTR11. |
| 13,597-14,561 | pCMR11 - specific; chloramphenicol resistance gene |
| | from pCC1FOS. |

Plasmid pCMR11 (SEQ ID NO 47) carries a chloramphenicol resistance gene. To convert this resistance gene to a kanamycin resistance marker, pCMR11 (SEQ ID NO 47) was digested with BspEI. The kanamycin resistance gene was amplified from pENTR11 (InVitrogen) with the tailed primers shown in Table 9 (SEQ ID NOs 48 and 49). The digested pCMR 11 (SEQ ID NO 47) and kanamycin amplicon were joined by recombination in yeast strain CMY1-5. The resulting plasmid was called pCMR10 (SEQ ID NO 50) (see Table 8).

TABLE 9

Tailed PCR primers used to amplify the kanamycin resistance marker
from pENTR11 for recombination into pCMR11.

| | |
|---|---|
| SEQ ID NO | Primer sequence |

| | |
|---|---|
| SEQ ID NO 48 | ATAAAATCATTATTTGCCATCCAGCTGCAGCTCTGGCCCGTGTCTCAAAATCTCTGATGTTACAT |
| SEQ ID NO 49 | TTTCTCTGTCCTTCCTGTGCGACGGTTACGCCGCTCCATGGTCTGACGCTCAGTGGAACGGGGCC |

Cloning and Assembly of CHK1sv1 Full-Length Clones and Yeast Transformation

In addition to sequence homology between the two sequences to be joined, the quantities of acceptor vector and donor PCR fragments are critical for efficient recombination. As described in Raymond et al. (2002, Genome Res. 12:190-197), 100 ng of acceptor vector and 1 μg of DNA donor fragment were used to transform yeast cells. Assembly of the full-length CHK1sv1 full length clone by homologous recombination between the 1243 base pair CHK1 amplicon, produced using the CHK1sv1 forward and reverse “tailed” primers described earlier, and the expression vector was performed by simultaneous transformation of these pieces into yeast cells. A subsequent recombination step with 80 base pair oligonucleotide linkers created the CHK1sv1 exon 9 to exon 11 splice junction. All yeast transformation steps described in subsequent paragraphs were performed by electroporation (Raymond et al., 2002 Genome Res. 12:190-197). Yeast transformation methods (electroporation, lithium acetate treatment, or spheroplasting) are also compared by Gietz and Woods (2001, Biotechniques 30:816-820, 822-826, 828).

1 μg of the 1243 base pair CHK1 purified amplicon was cloned directly into 100 ng of SrfI-digested pCMR11 (SEQ ID NO 47) by cotransformation of 100 μl of yeast strain CMY1-5 (Matα, URA3Δ, CYH2^{R}). Ura⁺, cycloheximide resistant colonies were selected on Ura-deficient media plates containing 1 μg/ml cycloheximide (Sigma, St. Louis, Mo.). Standard yeast media were used (Sherman, 1991, Methods Enzymol. 194:3-21). Total DNA from yeast cell culture containing the CHK1 clone was used to transform E. coli to chloramphenicol (Sigma, St. Louis, Mo.) resistance to prepare a large quantity of the recombinant plasmid as described in Hoffman and Winston (1987 Gene 57:267-72). The colonies were picked from the plates into 2 ml of 2× LB media. These liquid cultures were incubated overnight at 37° C. Plasmid DNA was extracted from these cultures using the Qiagen (Valencia, Calif.) Qiaquik Spin Miniprep it.

To construct the CHK1sv1 clone, 1 μg of 80 base pair linkers shown in Table 10 (SEQ ID NOs 51-52) that spans the region of the alternative splicing of exon 9 to exon 11, and 100 ng of BamHI-digested CHK1/pCMR11 clone were used to cotransform 100 μl of CMY1-5 yeast strain. The overlapping DNA between the linkers and CHK1/pCMR11 clone dictates that most yeast transformants will possess the correctly assembled construct. Ura⁺, cycloheximide resistant colonies were selected for subsequent preparation and transformation of E. coli. Plasmid DNA extracted from E. coli was analyzed by restriction digest to confirm the presence of the alternative splicing of exon 9 to exon 11 in the CHK1sv1 clone. Eight CHK1sv1 clones were sequenced to confirm identity, and the clones possessing the appropriate sequences are used for protein expression in multiple systems.

TABLE 10
Linkers used to create exon 9 to exon 11 splice junction for CHK1sv1 clone

| | |
|---|---|
| SEQ ID NO | Linker Sequence |

| | |
|---|---|
| SEQ ID NO 51 | AATCCAATTTGGACTTCTCTCCAGTAAACAGTGCTTCTAGAACCCCTGGCAGCGGTTGGTCA |
| | AAAGAATGACACGATTCT |
| SEQ ID NO 52 | AGAATCGTGTCATTCTTTTGACCAACCGCTGCCAGGGGTTCTAGAAGCACTGTTTACTGGAG |
| | AGAAGTCCAAATTGGATT |

Cloning and Assembly of CHK1sv2 Full-Length Clones and Yeast Transformation

Clones having a nucleotide sequence comprising the CHK1sv2 splice variant identified in Example 2 are isolated using recombination-mediated plasmid construction in yeast. A set of two primer pairs is used to amplify and clone the entire mRNA coding sequences of CHK1sv2. A 5′ “forward” primer and a 3′ “reverse” primer are designed for isolation of full length clones corresponding to CHK1sv2. The 5′ “forward” CHK1sv2 primer is designed to have the nucleotide sequence of 5′ TTACTGGCTTATCGAAATTAATACGACTCAC TATAG GGAGAGCATTTGTCTCCCACCTCATCATA 3′ (SEQ ID NO 53) and to have sequences complementary to exon 1 of the CHK1 mRNA (NM_{—}001274). The 3′ “reverse” CHK1sv2 primer is designed to have the nucleotide sequence of 5′ TAGAAGGCACAGTCGAGGCTG ATCAGCGGGTTTAAACTCAATTTGCAGTTTGCAGGACAGGATAA 3′ (SEQ ID NO 54) and to have sequences complementary to exon 13 of the CHK1 mRNA (NM_{—}001274). The 40 nucleotides at the 5′ ends of the primer sequences indicated in italics are “tails” that are incorporated into the PCR amplicons and facilitate subsequent plasmid recombination events in yeast.

To construct the CHK1sv2 clone, RT-PCR is performed as previously described using CHK1sv2 “forward” and “reverse” primers (SEQ ID NOs 53-54) on MOLT-4 mRNA. Nucleic acid bands in the gel are visualized and photographed on a UV light box to determine if the PCR yielded products of the expected size, in the case of the CHK1sv2 mRNA, a product of about 1521 base pairs. The purified amplicon is cloned directly into SrfI-digested pCMR11 (SEQ ID NO 47) by cotransformation of 100 μl of yeast strain CMY1-5 (Matα, URA3A, CYH2^{R}) as previously described. Ura⁺, cycloheximide resistant colonies are selected for subsequent preparation and transformation of E. coli. Plasmid DNA extracted from E. coli is analyzed by restriction digest to confirm the presence of the alternative splicing of exon 2 to exon 4 in the CHK1sv2 clone. Eight CHK1sv2 clones are sequenced to confirm identity, and the clones possessing the appropriate sequences are used for protein expression in multiple systems.

Summary of CHK1sv1 and CHK1sv2 Polynucleotides

The polynucleotide sequence of CHK1sv1 mRNA (SEQ ID NO 3) contains an open reading frame that encodes a CHK1sv1 protein (SEQ ID NO 4) similar to the reference CHK1 protein (NP_{—}001265), but lacking amino acids encoded by a 178 base pair region corresponding to exons 10 of the full length coding sequence of reference CHK1 mRNA (NM_{—}001274). The deletion of the 178 base pair region results in a shift of the protein translation reading frame in comparison to the reference CHK1 protein reading frame, creating a carboxy terminal peptide region that is unique to CHK1sv1. The frameshift also creates a premature termination codon 29 nucleotides downstream of the exon 9/exon 11 splice junction. Therefore, the CHK1sv1 protein is missing an internal 59 amino acid region corresponding to the amino acid region encoded by exon 10 and is also lacking the amino acids encoded by the nucleotides downstream of the premature stop codon as compared to the reference CHK1 (NP_{—}001265). Exon 10 encodes the SQ/TQ domains of CHK1, and exons 11-13 encode the autoinhibitory region (Sanchez et al., 1997, Science 277:1497-1501; Katsuragi and Sagata, 2004, Mol. Biol. Cell. 15:1680-1689). While deletion of the autoinhibitory region confers constitutive activity to the CHK1 kinase domain, when the SQ/TQ domains are also removed, CHK1 enzymatic activity decreases (Ng et al., 2004, J. Biol. Chem. 279:8808-8819).

The polynucleotide sequence of CHK1sv2 mRNA (SEQ ID NO 5) contains an open reading frame that encodes a CHK1sv2 protein (SEQ ID NO 6) similar to the reference CHK1 protein (NP_{—}001265), but lacking amino acids encoded by a 224 base pair region corresponding to exon 3 of the full length coding sequence of the reference CHK1 mRNA (NM_{—}001274). Furthermore, CHK1sv2 polypeptide initiates at an upstream AUG located 132 nucleotides from the beginning of exon 1. The use of the upstream AUG site and the deletion of the 224 base pair region corresponding to exon 3 change the protein translation reading frame in comparison to the reference CHK1 protein reading frame. Therefore, the CHK1sv2 protein has a unique N-terminal peptide region (amino acids 1-30) as compared to the reference CHK1 (NP_{—}001265). Exons 2 through 8 encode the kinase domain (Sanchez et al., 1997, Science 277:1497-1501; Chen et al., 2000, Cell 100:681-692). CHK1sv2 polypeptide is predicted to lack a functional kinase domain, but still possess an intact regulatory region. A rat liver isoform of CHK1 with a disrupted kinase domain and normal regulatory region acted as a dominant-negative inhibitor of normal CHK1 function (Shann and Hsu, 2001, J. Biol. Chem. 276:48863-48870).

### Example 4

### Expression of CHK1sv1 Protein

The baculovirus gene expression vector system permits protein expression insect cells, which are inexpensive and easy to maintain. The proteins produced are of similar quality to that in mammalian cells (Miller, 1988, Biotechnology 10:457-465; Miller, 1989, Bioessays 11:91-95). Methods of protein expression using the baculovirus expression vectors in insect cells are known in the art and techniques are discussed in O'Reilly et al., Baculovirus Expression Vectors—A Laboratory Manual, W. H. Freeman and Co., New York, 1992 and Baculovirus Expression Vector System Instruction Manual, 6^{th} edition, Pharmingen, San Diego, 1999.

Cloning CHK1sv1 for Insect Cell Expression

To create a CHK1sv1/baculovirus transfer vector construct, the CHK1sv1/pCMR11 clone (see Example 3) was used as template for PCR to amplify the coding sequence of CHK1sv1 (SEQ ID NO 3) using the primers listed in Table 11 (SEQ ID NOs 55, 56). The primer represented by SEQ ID NO 55 contains an optimal translation initiation sequence immediately upstream of the ATG start codon and an upstream EcoRI restriction site that become incorporated into the amplicon. The primer represented by SEQ ID NO 56 contains sequence encoding six histidine residues C-terminal to the CHK1sv1 coding sequence as well as an EagI restriction site that become incorporated into the CHK1sv1 amplicon. The CHK1sv1 amplicon was run on a 1% agarose gel. A selected amplicon fragment of the expected size, in the case of CHK1sv1, a product of about 994 base pairs, was manually extracted from the gel and purified with a Qiagen Gel Extraction Kit. The purified amplicon fragment was digested with EcoRI and EagI. The EcoRI/EagI-digested amplicon was ligated into the baculovirus transfer vector pVL1393 (Pharmingen, San Diego, Calif.) which had been digested with EcoRI and EagI and dephosphorylated with alkaline phosphatase. The CHK1sv1/pVL1393 construct was then transformed into E. coli strain DH5α. Plasmid DNA extracted from selected from ampicillin resistant colonies was sequenced to confirm identity, and the clones possessing the appropriate sequences were used for protein expression in insect cells.

TABLE 11

Primers used to clone CHK1sv1 into
baculovirus transfer vector pVL1393

| | |
|---|---|
| SEQ ID NO | Primer Sequence |

| | |
|---|---|
| SEQ ID NO 55 | CCCGGAATTCACCATGGCAGTGCCCTTTGTGGAAGAC |
| | TGG |
| SEQ ID NO 56 | TGTGTCCGGCCGTCAGTGATGGTGATGGTGATGTTCT |
| | TTTGACCAACCGCTGCC |

Insect Cell Expression of CHK1sv1

The CHK1sv1/pVL 1393 construct was co-transfected with linearized AcNPV BaculoGold DNA (Pharmingen, San Diego, Calif.) into SF9 insect cells (Invitrogen, Carlsbad, Calif.). Individual recombinant viruses were selected by end point dilution. Virus clones were amplified to obtain high titer stocks. These virus stocks were used for protein expression tests in small scale SF9 cultures to verify production of the CHK1sv1 recombinant protein. Transfected SF9 cell lysates were analyzed by polyacrylamide gel electrophoresis for CHK1sv1 protein expression. The CHK1sv1 protein was visualized by Commassie staining or by Western blotting using an anti-CHK1 antibody (G4 antibody; Santa Cruz Biotechnology, Inc). Based on expression, an individual virus was selected for larger scale CHK1sv1 expression. For recombinant protein expression on the liter scale, SF9 suspension cultures were grown at 27° C. in Ex-cell 401 serum-free media (JRH Scientific, Lenexa, Kans.) and were infected with a recombinant virus stock using a multiplicity of infection of 0.3 virus per cell. The infected SF9 culture was harvested 72 hour following virus transfection, and pelleted by centrifugation. Pellets were stored at −70° C.

Purification of CHK1sv1 Recombinant Protein

Insect cell pellets were lysed with B-PER protein extraction reagent (Pierce, Rockford, Ill.) containing 1 μM microcystin (Sigma), 10 μM cypermethrin (CalBiochem), and EDTA-free Protease Inhibitor Cocktail (1 tablet/50 ml lysis buffer). All manipulations during protein purification were performed at 4° C. Cells were resuspended in the lysis buffer were stirred for 45 minutes. DNAseI (Roche) was then added to a final concentration of 200 U/ml and the cell suspension was stirred for an additional 30 minutes. The lysed cell suspension was centrifuged for 30 minutes at 30,000 g. The lysis supernatant was decanted and centrifuged for 30 minutes at 30,000 g. For each 10 ml of cleared supernatant, 1 ml bed volume of Talon metal affinity resin (Clontech, Palo Alto, Calif.) was added, and the suspension was stirred for 45 minutes. The affinity resin/lysate suspension was centrifuged at 5000 g for 3 minutes and then the supernatant was discarded. The affinity resin was washed 4× with Buffer A (50 μM Tris, pH 8.0; 250 mM NaCl) using 5× volumes of the resin. The washed resin was resuspended as a 2× slurry in Buffer A and packed into a chromatography column. The resin-packed column was washed with 6× bed volumes of Buffer A. CHK1sv1-His-tagged protein is eluted from the column using a step-wise gradient of imidazole in Buffer A. Imidazole concentrations in the 2× bed volumen fractions were 5, 10, 20, 30, 40, 50, and 60 mM. Elution fractions were concentrated using the Amicon Ultra 15 Centrifugal Filter Device, 30,000 Nominal Molecular Weight Limit (Millipore, Billerica, Mass.). The concentrated enzyme fractions were diluted 50% in glycerol and stored at −20° C. Fractions were analyzed for the presence of CHK1sv1-His-tagged protein using polyacrylamide gel electrophoresis followed by Coommassie staining and Western blotting using an anti-CHK1 antibody (G4 antibody; Santa Cruz Biotechnology, Inc). The CHK1sv1 kinase activity of the column fractions was determined using the kinase assay described in the following section.

CHK1sv1 Kinase Assay

CHK1sv1 activity was assayed in vitro using a synthetic peptide substrate. The phophopeptide product was quantitated using a Homogenous Time-Resolved Fluorescence assay system (Park et al., 1999, Anal. Biochem. 269:94-104). The reaction mixture contained 40 mM HEPES, pH 7.3; 100 mM NaCl; 10 mM MgCl₂; 2 mM dithiothreitol; 0.1% BSA; 0.1 mM ATP; 0.5 μM peptide substrate; and 0.1 nM CHK1sv1 enzyme in a final volume of 40 μl. The peptide substrate has the amino acid sequence amino terminus-GGRARTSSFAEPG-carboxy terminus (SynPep, Dublin Calif.) (SEQ ID NO 57) and is biotinylated at the N-terminus. The kinase reaction was incubated for 30 minutes at 22° C., and then terminated with 60 μl Stop/Detection Buffer (40 mM HEPES, pH 7.3; 10 mM EDTA; 0.125% Triton X-100; 1.25% BSA; 250 nM PhycoLink Streptavidin-Allophycocyanin (APC) Conjugate (Prozyme, San Leandro, Calif.); and 0.75 mM Europium-chelate labeled anti-phosphotyrosine antibody (PY20, Perkin Elmer, Boston, Mass.)). The reaction was allowed to equilibrate for 2 hours at 22° C., and relative fluorescent units were read on a Discovery plate reader (Packard Biosciences). ATP Kₘ was determined by varying ATP concentration from 0.003 to 3 mM in the reaction described above and determining the relative enzymatic rates. Peptide substrate Kₘ was determined by varying the concentration of a peptide derived from the known CHK1 kinase substrate CDC25C (amino terminus-GLYRSPSMPENLNR-carboxy terminus) (SynPep, Dublin, Calif.) (SEQ ID NO 58) from 0.0003 to 1 mM and determining the relative enzymatic rates under the reaction conditions described above. CHK1sv1 Vₘₐₓ was calculated by determining the amount of phospho-peptide product formed per microgram of CHKsv1 when both ATP and CDC25C peptide substrate are at saturating concentrations. To titrate the known kinase inhibitor staurosporin (Calbiochem, San Diego, Calif.) against CHK1sv1, 1 μl of the inhibitor from a 40× serial dilution series in DMSO was added to the kinase reaction mixture described above, prior to addition of CHK1sv1. The the CHK1sv1 Vₘₐₓ, Kₘ, and staurosporin IC₅₀ value are given in Table 12 along with the values for the reference CHK1 that were previously described (O'Neill et al., 2002, J. Biol. Chem. 277:16102-16115; Jackson et al., 2000, Cancer Res. 60:566-572). Additional staurosporin derivative kinase inhibitors (Calbiochem, San Diego, Calif.); including K252A, RO-320432, RO317549, and Gö6976; were titrated against CHK1sv1 as described above. Table 13 presents their IC₅₀ values as well as the IC₅₀ value for the reference CHK1 inhibited by Gö6976 that was previously published (Davies et al., 2000, Biochem. J. 351:95-105).

TABLE 12
Comparison of CHK1sv1 and reference CHK1 activity and
inhibition by staurosporin

| | Vₘₐₓ | Kₘ (CDC25C substrate) | IC₅₀ |
|---|---|---|---|
| CHK1sv1 | 36 nmol/min/μg | 120 μM | 0.00013 μM |
| CHK1 | 0.6 nmol/min/μg | 12 μM | 0.008 μM |

TABLE 13
CHK1sv1 inhibition by derivatives of staurosporin, IC₅₀ values

| | K252A | RO-320432 | RO317549 | Gö6976 |
|---|---|---|---|---|
| CHK1sv1 | 0.05 μM | 0.39 μM | 0.125 μM | 0.00075 μM |
| CHK1 | | | | 0.003 μM |

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

1. A purified human nucleic acid comprising SEQ ID NO 3, or the complement thereof.

2. The purified nucleic acid of claim 1, wherein said nucleic acid comprises a region encoding SEQ ID NO 4.

3. The purified nucleic acid of claim 1, wherein said nucleotide sequence encodes a polypeptide consisting of SEQ ID NO 4.

4. A purified polypeptide comprising SEQ ID NO 4.

5. The polypeptide of claim 4, wherein said polypeptide consists of SEQ ID NO 4.

6. An expression vector comprising a nucleotide sequence encoding SEQ ID NO 4, wherein said nucleotide sequence is transcriptionally coupled to an exogenous promoter.

7. The expression vector of claim 6, wherein said nucleotide sequence encodes a polypeptide consisting of SEQ ID NO 4.

8. The expression vector of claim 6, wherein said nucleotide sequence comprises SEQ ID NO 3.

9. The expression vector of claim 6, wherein said nucleotide sequence consists of SEQ ID NO 3.

10. A method of screening for compounds able to bind selectively to CHK1sv1 comprising the steps of:
(a) providing a CHK1sv1 polypeptide comprising SEQ ID NO 4;
(b) providing one or more CHK1 isoform polypeptides that are not CHK1sv1;
(c) contacting said CHK1sv1 polypeptide and said CHK1 isoform polypeptide that is not CHK1sv1 with a test preparation comprising one or more compounds; and
(d) determining the binding of said test preparation to said CHK1sv1 polypeptide and to said CHK1 isoform polypeptide that is not CHK1sv1, wherein a test preparation that binds to said CHK1sv1 polypeptide, but does not bind to said CHK1 polypeptide that is not CHK1sv1, contains a compound that selectively binds said CHK1sv1 polypeptide.

11. The method of claim 10, wherein said CHK1sv1 polypeptide is obtained by expression of said polypeptide from an expression vector comprising a polynucleotide encoding SEQ ID NO 4.

12. The method of claim 11, wherein said polypeptide consists of SEQ ID NO 4.

13. A method for screening for a compound able to bind to or interact with a CHK1sv1 protein or a fragment thereof comprising the steps of:
(a) expressing a CHK1sv1 polypeptide comprising SEQ ID NO 4 or fragment thereof from a recombinant nucleic acid;
(b) providing to said polypeptide a labeled CHK1 ligand that binds to said polypeptide and a test preparation comprising one or more compounds; and
(c) measuring the effect of said test preparation on binding of said labeled CHK1 ligand to said polypeptide, wherein a test preparation that alters the binding of said labeled CHK1 ligand to said polypeptide contains a compound that binds to or interacts with said polypeptide.

14. The method of claim 13, wherein said steps (b) and (c) are performed in vitro.

15. The method of claim 13, wherein said steps (a), (b) and (c) are performed using a whole cell.

16. The method of claim 13, wherein said polypeptide is expressed from an expression vector.

17. The method of claim 13, wherein said CHK1sv1 ligand is a CHK1 inhibitor.

18. The method of claim 16, wherein said expression vector comprises SEQ ID NO 3 or a fragment of SEQ ID NO 3.

19. The method of claim 16, wherein said polypeptide comprises SEQ ID NO 4 or a fragment of SEQ ID NO 4.

20. The method of claim 16, wherein said test preparation contains one compound.

21. A method of screening for CHK1sv1 activity comprising the steps of:
(a) contacting a cell expressing a recombinant nucleic acid encoding CHK1sv1 comprising SEQ ID NO 4 with a test preparation comprising one or more test compounds; and
(b) measuring the effect of said test preparation on protein kinase activity.

22. A method of screening for compounds able to bind selectively to one or more CHK1 isoform polypeptides but not to CHK1sv1 comprising the steps of:
(a) providing a CHK1 isoform polypeptide that is not CHKsv1;
(b) providing a CHK1sv1 polypeptide comprising SEQ ID NO 4;
(c) contacting said CHK1 isoform polypeptide that is not CHKsv1 and said CHKsv1 polypeptide with a test preparation comprising one or more compounds; and
(d) determining the binding of said test preparation to said CHK1 isoform polypeptide that is not CHK1sv1 and to said CHK1sv1 polypeptide, wherein a test preparation that binds to said CHK1 isoform polypeptide that is not CHK1sv1, but does not bind to CHK1sv1 polypeptide, contains a compound that selectively binds said CHK1 isoform polypeptide that is not CHK1sv1.

23. A method for screening compounds able to inhibit or alter the interaction between CHK1sv1 polypeptide and CHK1 ligands comprising the steps of:
(a) providing a CHK1sv1 polypeptide comprising SEQ ID NO 4 or fragment thereof;
(b) providing to said polypeptide a CHK1 ligand that binds to said polypeptide and a test preparation comprising one or more compounds; and
(c) measuring the effect of said test preparation on the interaction of said CHK1 ligand with said CHK1sv1 polypeptide, wherein a test preparation that alters the interaction between said CHK1 ligand with said CHK1sv1 polypeptide contains a compound that inhibits or alters the interaction between said CHK1sv1 polypeptide and said CHK1 ligand.

24. The method of claim 23, wherein said CHK1 ligand is selected from the group consisting of: CDC25, p53, and TLK1/2.

25. A method for screening compounds able to modulate the kinase activity of CHK1sv1 comprising the steps of:
(a) providing a CHK1sv1 polypeptide comprising SEQ ID NO 4 or fragment thereof;
(b) providing to said CHK1sv1 polypeptide a kinase substrate and a test preparation comprising one or more compounds; and
(c) determining the effect of said test preparation on protein kinase activity of said CHK1sv1 polypeptide to thereby identify a compound that modulates the activity of the said CHK1sv1 polypeptide.

26. The method of claim 25, wherein said kinase substrate is selected from the group consisting of: CDC25, p53, and TLK1/2.


SEQUENCE LISTING (58 sequences)

SEQ ID NO: 1 - DNA, 40 bp, Homo sapiens
ccagtaaacagtgcttctagaacccctggcagcggttggt

SEQ ID NO: 2 - DNA, 40 bp, Homo sapiens
ggagaaggtgcctatggagaagccagacataggcatgcct

SEQ ID NO: 3 - DNA, 951 bp, Homo sapiens
atggcagtgccctttgtggaagactgggacttggtgcaaaccctgggagaaggtgcctatggagaagttcaacttgctgtgaatagagtaactgaagaagcagtcgcagtgaagattgtagatatgaagcgtgccgtagactgtccagaaaatattaagaaagagatctgtatcaataaaatgctaaatcatgaaaatgtagtaaaattctatggtcacaggagagaaggcaatatccaatatttatttctggagtactgtagtggaggagagctttttgacagaatagagccagacataggcatgcctgaaccagatgctcagagattcttccatcaactcatggcaggggtggtttatctgcatggtattggaataactcacagggatattaaaccagaaaatcttctgttggatgaaagggataacctcaaaatctcagactttggcttggcaacagtatttcggtataataatcgtgagcgtttgttgaacaagatgtgtggtactttaccatatgttgctccagaacttctgaagagaagagaatttcatgcagaaccagttgatgtttggtcctgtggaatagtacttactgcaatgctcgctggagaattgccatgggaccaacccagtgacagctgtcaggagtattctgactggaaagaaaaaaaaacatacctcaacccttggaaaaaaatcgattctgctcctctagctctgctgcataaaatcttagttgagaatccatcagcaagaattaccattccagacatcaaaaaagatagatggtacaacaaacccctcaagaaaggggcaaaaaggccccgagtcacttcaggtggtgtgtcagagtctcccagtggattttctaagcacattcaatccaatttggacttctctccagtaaacagtgcttctagaacccctggcagcggttggtcaaaagaa

SEQ ID NO: 4 - protein, 317 aa, Homo sapiens
MAVPFVEDWDLVQTLGEGAYGEVQLAVNRVTEEAVAVKIVDMKRAVDCPENIKKEICINKMLNHENVVKFYGHRREGNIQYLFLEYCSGGELFDRIEPDIGMPEPDAQRFFHQLMAGVVYLHGIGITHRDIKPENLLLDERDNLKISDFGLATVFRYNNRERLLNKMCGTLPYVAPELLKRREFHAEPVDVWSCGIVLTAMLAGELPWDQPSDSCQEYSDWKEKKTYLNPWKKIDSAPLALLHKILVENPSARITIPDIKKDRWYNKPLKKGAKRPRVTSGGVSESPSGFSKHIQSNLDFSPVNSASRTPGSGWSKE

SEQ ID NO: 5 - DNA, 1227 bp, Homo sapiens
atgccgaggtgctcggtggagtcatggcagtgccctttgtggaagactgggacttggtgcaaaccctgggagaaggtgcctatggagaagccagacataggcatgcctgaaccagatgctcagagattcttccatcaactcatggcaggggtggtttatctgcatggtattggaataactcacagggatattaaaccagaaaatcttctgttggatgaaagggataacctcaaaatctcagactttggcttggcaacagtatttcggtataataatcgtgagcgtttgttgaacaagatgtgtggtactttaccatatgttgctccagaacttctgaagagaagagaatttcatgcagaaccagttgatgtttggtcctgtggaatagtacttactgcaatgctcgctggagaattgccatgggaccaacccagtgacagctgtcaggagtattctgactggaaagaaaaaaaaacatacctcaacccttggaaaaaaatcgattctgctcctctagctctgctgcataaaatcttagttgagaatccatcagcaagaattaccattccagacatcaaaaaagatagatggtacaacaaacccctcaagaaaggggcaaaaaggccccgagtcacttcaggtggtgtgtcagagtctcccagtggattttctaagcacattcaatccaatttggacttctctccagtaaacagtgcttctagtgaagaaaatgtgaagtactccagttctcagccagaaccccgcacaggtctttccttatgggataccagcccctcatacattgataaattggtacaagggatcagcttttcccagcccacatgtcctgatcatatgcttttgaatagtcagttacttggcaccccaggatcctcacagaacccctggcagcggttggtcaaaagaatgacacgattctttaccaaattggatgcagacaaatcttatcaatgcctgaaagagacttgtgagaagttgggctatcaatggaagaaaagttgtatgaatcaggttactatatcaacaactgataggagaaacaataaactcattttcaaagtgaatttgttagaaatggatgataaaatattggttgacttccggctttctaagggtgatggattggagttcaagagacacttcctgaagattaaagggaagctgattgatattgtgagcagccagaaggtttggcttcctgccaca

SEQ ID NO: 6 - protein, 409 aa, Homo sapiens
MPRCSVESWQCPLWKTGTWCKPWEKVPMEKPDIGMPEPDAQRFFHQLMAGVVYLHGIGITHRDIKPENLLLDERDNLKISDFGLATVFRYNNRERLLNKMCGTLPYVAPELLKRREFHAEPVDVWSCGIVLTAMLAGELPWDQPSDSCQEYSDWKEKKTYLNPWKKIDSAPLALLHKILVENPSARITIPDIKKDRWYNKPLKKGAKRPRVTSGGVSESPSGFSKHIQSNLDFSPVNSASSEENVKYSSSQPEPRTGLSLWDTSPSYIDKLVQGISFSQPTCPDHMLLNSQLLGTPGSSQNPWQRLVKRMTRFFTKLDADKSYQCLKETCEKLGYQWKKSCMNQVTISTTDRRNNKLIFKVNLLEMDDKILVDFRLSKGDGLEFKRHFLKIKGKLIDIVSSQKVWLPAT

SEQ ID NO: 7 - DNA, 20 bp, Artificial Sequence
  Oligonucleotide probe
gtgcttctagaacccctggc

SEQ ID NO: 8 - DNA, 20 bp, Artificial Sequence
  Oligonucleotide probe
cctatggagaagccagacat

SEQ ID NO: 9 - protein, 10 aa, Homo sapiens
VNSASRTPGS

SEQ ID NO: 10 - protein, 10 aa, Homo sapiens
KVPMEKPDIG

SEQ ID NO: 11 - DNA, 28 bp, Artificial Sequence
  Oligonucleotide
atcagcaagaattaccattccagacatc

SEQ ID NO: 12 - DNA, 28 bp, Artificial Sequence
  Oligonucleotide
catacaacttttcttccattgatagccc

SEQ ID NO: 13 - DNA, 19 bp, Artificial Sequence
  Oligonucleotide
gttacttggcaccccagga

SEQ ID NO: 14 - DNA, 27 bp, Artificial Sequence
  Oligonucleotide
catccaatttggtaaagaatcgtgtca

SEQ ID NO: 15 - DNA, 15 bp, Artificial Sequence
  Oligonucleotide probe
tcctcacagaacccc

SEQ ID NO: 16 - DNA, 26 bp, Artificial Sequence
  Oligonucleotide
gcacattcaatccaatttggacttct

SEQ ID NO: 17 - DNA, 28 bp, Artificial Sequence
  Oligonucleotide
catccaatttggtaaagaatcgtgtcat

SEQ ID NO: 18 - DNA, 17 bp, Artificial Sequence
  Oligonucleotide probe
cagtgcttctagaaccc

SEQ ID NO: 19 - DNA, 24 bp, Artificial Sequence
  Oligonucleotide
tgcatccaatttggtaaagaatcg

SEQ ID NO: 20 - DNA, 22 bp, Artificial Sequence
  Oligonucleotide
gagtcatggcagtgccctttgt

SEQ ID NO: 21 - DNA, 62 bp, Artificial Sequence
  Oligonucleotide
ttactggcttatcgaaattaatacgactcactatagggaggagtcatggcagtgccctttgt

SEQ ID NO: 22 - DNA, 64 bp, Artificial Sequence
  Oligonucleotide
tagaaggcacagtcgaggctgatcagcgggtttaaactcatgcatccaatttggtaaagaatcg

SEQ ID NO: 23 - DNA, 64 bp, Artificial Sequence
  Oligonucleotide
atacgactcactatagggagacccaagctggctagttaagtatgtttatatatggattttgaaa

SEQ ID NO: 24 - DNA, 41 bp, Artificial Sequence
  Oligonucleotide
gtagaggtatggccggtggtgaacatcaccacagaattaac

SEQ ID NO: 25 - DNA, 73 bp, Artificial Sequence
  Oligonucleotide
tccaccacactggactagtggatccgagctcggtaccaaggcccgggcatgctacgtacctgtttaactcttc

SEQ ID NO: 26 - DNA, 41 bp, Artificial Sequence
  Oligonucleotide
gttaattctgtggtgatgttcaccaccggccatacctctac

SEQ ID NO: 27 - DNA, 80 bp, Artificial Sequence
  Oligonucleotide linker
tgcgttatcccctgattctgtggataaccgtattaccgctgggtaataactgatataattaaattgaagctctaatttgt

SEQ ID NO: 28 - DNA, 80 bp, Artificial Sequence
  Oligonucleotide linker
acaaattagagcttcaatttaattatatcagttattacccagcggtaatacggttatccacagaatcaggggataacgca

SEQ ID NO: 29 - DNA, 80 bp, Artificial Sequence
  Oligonucleotide linker
aatttaaattataattatttttatagcacgtgatgaaaagagcatggatctcggggacgtctaactactaagcgagagta

SEQ ID NO: 30 - DNA, 80 bp, Artificial Sequence
  Oligonucleotide linker
tactctcgcttagtagttagacgtccccgagatccatgctcttttcatcacgtgctataaaaataattataatttaaatt

SEQ ID NO: 31 - DNA, 4615 bp, Artificial Sequence
  vector
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggcttcgaaggagatagaaccaattctctaaggaaatacttaaccatggtcgactggatccggtaccgaattcgcttactaaaagccagataacagtatgcgtatttgcgcgctgatttttgcggtataagaatatatactgatatgtatacccgaagtatgtcaaaaagaggtgtgcttctagaatgcagtttaaggtttacacctataaaagagagagccgttatcgtctgtttgtggatgtacagagtgatattattgacacgcccgggcgacggatagtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatatagaattcgcggccgcactcgagatatctagacccagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgct

SEQ ID NO: 32 - DNA, 88 bp, Artificial Sequence
  Oligonucleotide linker
actttgtacaaaaaagcaggcttcgaaggagatagaaccagcccgggcgccgcactcgagatatctagacccagctttcttgtacaaa

SEQ ID NO: 33 - DNA, 88 bp, Artificial Sequence
  Oligonucleotide linker
tttgtacaagaaagctgggtctagatatctcgagtgcggcgcccgggctggttctatctccttcgaagcctgcttttttgtacaaagt

SEQ ID NO: 34 - DNA, 4146 bp, Artificial Sequence
  vector
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggcttcgaaggagatagaaccagcccgggcgccgcactcgagatatctagacccagctttcttgtacaaagttggcattataagaaagcattgcttatcaatttgttgcaacgaacaggtcactatcagtcaaaataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgct

SEQ ID NO: 35 - DNA, 80 bp, Artificial Sequence
  Oligonucleotide linker
gttaaccgggctgcatccgatgcaagtgtgtcgctgtcgagggtaataactgatataattaaattgaagctctaatttgt

SEQ ID NO: 36 - DNA, 80 bp, Artificial Sequence
  Oligonucleotide linker
acaaattagagcttcaatttaattatatcagttattaccctcgacagcgacacacttgcatcggatgcagcccggttaac

SEQ ID NO: 37 - DNA, 80 bp, Artificial Sequence
  Oligonucleotide linker
ataaaatcattatttgccatccagctgcagctctggcccgtcgaatttctgccattcatccgcttattatcacttattca

SEQ ID NO: 38 - DNA, 80 bp, Artificial Sequence
  Oligonucleotide linker
tgaataagtgataataagcggatgaatggcagaaattcgacgggccagagctgcagctggatggcaaataatgattttat

SEQ ID NO: 39 - DNA, 9430 bp, Artificial Sequence
  vector
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagagcatggatctcggggacgtctaactactaagcgagagtagggaactgccaggcatcaaataaaacgaaaggctcagtcggaagactgggcctttcgttttatctgttgtttgtcggtgaacgctctcctgagtaggacaaatccgccgggagcggatttgaacgttgtgaagcaacggcccggagggtggcgggcaggacgcccgccataaactgccaggcatcaaactaagcagaaggccatcctgacggatggcctttttgcgtttctacaaactcttcctgttagttagttacttaagctcgggccccaaataatgattttattttgactgatagtgacctgttcgttgcaacaaattgataagcaatgcttttttataatgccaactttgtacaaaaaagcaggcttcgaaggagatagaaccagcccgggcgccgcactcgagatatctagacccagctttcttgtacaaagttggcattataagaaagcattgcttatcaatttgttgcaacgaacaggtcactatcagtcaaaataaaatcattatttgccatccagctgcagctctggcccgtcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcga

SEQ ID NO: 40 - DNA, 80 bp, Artificial Sequence
  Oligonucleotide linker
acggttatccacagaatcaggggataacgcaggaaagaacataaaatcattatttgccatccagctgcagctctggcccg

SEQ ID NO: 41 - DNA, 80 bp, Artificial Sequence
  Oligonucleotide linker
cgggccagagctgcagctggatggcaaataatgattttatgttctttcctgcgttatcccctgattctgtggataaccgt

SEQ ID NO: 42 - DNA, 80 bp, Artificial Sequence
  Oligonucleotide linker
aatttaaattataattatttttatagcacgtgatgaaaagtccgcgcacatttccccgaaaagtgccacctgacgtcgac

SEQ ID NO: 43 - DNA, 80 bp, Artificial Sequence
  Oligonucleotide linker
gtcgacgtcaggtggcacttttcggggaaatgtgcgcggacttttcatcacgtgctataaaaataattataatttaaatt

SEQ ID NO: 44 - DNA, 12604 bp, Artificial Sequence
  vector
gggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagtccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagcttggtaccgagctcggatccactagtccagtgtggtggaattctgcagatatccagcacagtggcggccgctcgagtctagagggcccttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacataaaatcattatttgccatccagctgcagctctggcccgtcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcga

SEQ ID NO: 45 - DNA, 41 bp, Artificial Sequence
  Oligonucleotide
gtagaggtatggccggtggtcaacatcaccacagaattaac

SEQ ID NO: 46 - DNA, 41 bp, Artificial Sequence
  Oligonucleotide
gttaattctgtggtgatgttgaccaccggccatacctctac

SEQ ID NO: 47 - DNA, 14561 bp, Artificial Sequence
  vector
catggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagtccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagtatgtttatatatggattttgaaaatcttttaaaaaagcgataaaagcgttgggatctgccactggtaacttcaaaatagacaagacgaaaaaacggtgaaaatgggtgatagaaataatacagaagtagatgttgaattagattaaactgaagatatataatttattggaaaatacatagagtttttgttgatgcgcttaagcgatcaattcaacaacaccaccagcagctctgattttttcttcagccaacttggagacgaatctagctttgacgataactggaacatttggaattctacccttacccaagatcttaccgtaaccggctgccaaagtgtcaataactggagcagtttccttagaagcagatttcaagtattggtctctcttgtcttctgggatcaatgtccacaatttgtccaagttcaagactggcttccagaaatgagcttgttgcttgtggaagtatctcataccaaccttaccgaaataacctggatggtatttatccatgttaattctgtggtgatgttcaccaccggccatacctctaccaccggggtgctttctgtgcttaccgatacgacctttaccggctgtacaaaaaaaatattgtaatgaaatacaatttttaaatcgttagtaaaacaggttttcaactggacattttatcgaaacaaaagaaacgtggtaactctttttcataattgcgctctctacaaccatttgatacagcgataattagtgctttccataatctaaatgaaagttaccttttatagccgtaaaatgagtagacagtatgactgttctgaatatcatggtataataaaggagaagtatgacaaaggctatgcacactttctttttgctcagtttgcgatggaagagtacatttactcgagctattacctcaaataagccgtctcaacagtgagatggtaatacaaagttcgcaatcaaaacacaaagaaatatgttgagtacacttagctgattgacattctcctgtaccatgatatacacacgacatattggttgcacaacactaaaaaaaaagtcaccgtcttggttctttcattccctcttccaaatggaactacatacctgagacgtgacctctgtgctttctagtcttagtgaatctggaaggcattcttgattagttggatgattgttctgggatttaatgcaaaaaaatcactaagaaggaaaaaaatcaacggagaaagcaaacgccatcttaaatatacgggatacagatgaaaggtttgaacctatctgggaaaatacgcattaaacaagcgaaaaactgcgaggaaaattgtttgcgtctctgcgggctattcacgcgccagaggaaaaataggaaaaataacaggcattagaaaaataattttgattttggtaatgtgtgggtccctggtgtacagatgttacattggttacagtactcttgtttttgctgtgtttttcgaaagggtttcctgggaactagtgctataatgtcaacatcaagtatcgtagatttcaattcaacttagctcttattacagtttatatataatacgcatttcataaggttcattccacctcgtctcttgtaatcattacaatgctgttgatcaactatatttaacaatattatagcttgtttctttataacattagcttacgctcataggcggcagtaaacgatgattaaccatgaaacgtaaggtaacggtggcataatttggtatttgttttctttttttcattttttgaactattacttggttttttcgagagcaaactacctcatacttatttcttgtgtgttaatattatattggcggcaaagtgaaaggggtgatcattctgttatgaaaaaaataaagaacttaagaggaacggtgaagaaagaaaacggataaaatgagaagagttaaacaggtacgtagcatgcccgggccttggtaccgagctcggatccactagtccagtgtggtggaattctgcagatatccagcacagtggcggccgctcgagtctagagggcccttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacataaaatcattatttgccatccagctgcagctctggcccgtcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagct

SEQ ID NO: 48 - DNA, 65 bp, Artificial Sequence
  Oligonucleotide
ataaaatcattatttgccatccagctgcagctctggcccgtgtctcaaaatctctgatgttacat

SEQ ID NO: 49 - DNA, 65 bp, Artificial Sequence
  Oligonucleotide
tttctctgtccttcctgtgcgacggttacgccgctccatggtctgacgctcagtggaacggggcc

SEQ ID NO: 50 - DNA, 14567 bp, Artificial Sequence
  vector
catggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgagggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagcgtggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttaggacggatcgcttgcctgtaacttacacgcgcctcgtatcttttaatgatggaataatttgggaatttactctgtgtttatttatttttatgttttgtatttggattttagaaagtaaataaagaaggtagaagagttacggaatgaagaaaaaaaaataaacaaaggtttaaaaaatttcaacaaaaagcgtactttacatatatatttattagacaagaaaagcagattaaatagatatacattcgattaacgataagtaaaatgtaaaatcacaggattttcgtgtgtggtcttctacacagacaagatgaaacaattcggcattaatacctgagagcaggaagagcaagataaaaggtagtatttgttggcgatccccctagagtcttttacatcttcggaaaacaaaaactattttttctttaatttctttttttactttctatttttaatttatatatttatattaaaaaatttaaattataattatttttatagcacgtgatgaaaagtccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagttaagtatgtttatatatggattttgaaaatcttttaaaaaagcgataaaagcgttgggatctgccactggtaacttcaaaatagacaagacgaaaaaacggtgaaaatgggtgatagaaataatacagaagtagatgttgaattagattaaactgaagatatataatttattggaaaatacatagagtttttgttgatgcgcttaagcgatcaattcaacaacaccaccagcagctctgattttttcttcagccaacttggagacgaatctagctttgacgataactggaacatttggaattctacccttacccaagatcttaccgtaaccggctgccaaagtgtcaataactggagcagtttccttagaagcagatttcaagtattggtctctcttgtcttctgggatcaatgtccacaatttgtccaagttcaagactggcttccagaaatgagcttgttgcttgtggaagtatctcataccaaccttaccgaaataacctggatggtatttatccatgttaattctgtggtgatgttcaccaccggccatacctctaccaccggggtgctttctgtgcttaccgatacgacctttaccggctgtacaaaaaaaatattgtaatgaaatacaatttttaaatcgttagtaaaacaggttttcaactggacattttatcgaaacaaaagaaacgtggtaactctttttcataattgcgctctctacaaccatttgatacagcgataattagtgctttccataatctaaatgaaagttaccttttatagccgtaaaatgagtagacagtatgactgttctgaatatcatggtataataaaggagaagtatgacaaaggctatgcacactttctttttgctcagtttgcgatggaagagtacatttactcgagctattacctcaaataagccgtctcaacagtgagatggtaatacaaagttcgcaatcaaaacacaaagaaatatgttgagtacacttagctgattgacattctcctgtaccatgatatacacacgacatattggttgcacaacactaaaaaaaaagtcaccgtcttggttctttcattccctcttccaaatggaactacatacctgagacgtgacctctgtgctttctagtcttagtgaatctggaaggcattcttgattagttggatgattgttctgggatttaatgcaaaaaaatcactaagaaggaaaaaaatcaacggagaaagcaaacgccatcttaaatatacgggatacagatgaaaggtttgaacctatctgggaaaatacgcattaaacaagcgaaaaactgcgaggaaaattgtttgcgtctctgcgggctattcacgcgccagaggaaaaataggaaaaataacaggcattagaaaaataattttgattttggtaatgtgtgggtccctggtgtacagatgttacattggttacagtactcttgtttttgctgtgtttttcgaaagggtttcctgggaactagtgctataatgtcaacatcaagtatcgtagatttcaattcaacttagctcttattacagtttatatataatacgcatttcataaggttcattccacctcgtctcttgtaatcattacaatgctgttgatcaactatatttaacaatattatagcttgtttctttataacattagcttacgctcataggcggcagtaaacgatgattaaccatgaaacgtaaggtaacggtggcataatttggtatttgttttctttttttcattttttgaactattacttggttttttcgagagcaaactacctcatacttatttcttgtgtgttaatattatattggcggcaaagtgaaaggggtgatcattctgttatgaaaaaaataaagaacttaagaggaacggtgaagaaagaaaacggataaaatgagaagagttaaacaggtacgtagcatgcccgggccttggtaccgagctcggatccactagtccagtgtggtggaattctgcagatatccagcacagtggcggccgctcgagtctagagggcccttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacataaaatcattatttgccatccagctgcagctctggcccgtgtctcaaaatctctgatgttacattgcacaagataaaaatatatcatcatgaacaataaaactgtctgcttacataaacagtaatacaaggggtgttatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacattattcagattgggccccgttccactgagcgtcagac

SEQ ID NO: 51 - DNA, 80 bp, Artificial Sequence
  Oligonucleotide linker
aatccaatttggacttctctccagtaaacagtgcttctagaacccctggcagcggttggtcaaaagaatgacacgattct

SEQ ID NO: 52 - DNA, 80 bp, Artificial Sequence
  Oligonucleotide linker
agaatcgtgtcattcttttgaccaaccgctgccaggggttctagaagcactgtttactggagagaagtccaaattggatt

SEQ ID NO: 53 - DNA, 65 bp, Artificial Sequence
  Oligonucleotide
ttactggcttatcgaaattaatacgactcactatagggagagcatttgtctcccacctcatcata

SEQ ID NO: 54 - DNA, 65 bp, Artificial Sequence
  Oligonucleotide
tagaaggcacagtcgaggctgatcagcgggtttaaactcaatttgcagtttgcaggacaggataa

SEQ ID NO: 55 - DNA, 40 bp, Artificial Sequence
  Oligonucleotide
cccggaattcaccatggcagtgccctttgtggaagactgg

SEQ ID NO: 56 - DNA, 54 bp, Artificial Sequence
  Oligonucleotide
tgtgtccggccgtcagtgatggtgatggtgatgttcttttgaccaaccgctgcc

SEQ ID NO: 57 - protein, 13 aa, Artificial Sequence
  synthetic peptide substrate for CHK1 activity assay
GGRARTSSFAEPG

SEQ ID NO: 58 - protein, 14 aa, Homo sapiens
GLYRSPSMPENLNR